(19) United States
(12) Patent Application Publication
(10) Pub. No.: US 2024/0069010 A1
(43) Pub. Date: Feb. 29, 2024

(54) TISSUE ORGANOID BIOPRINTING AND HIGH-THROUGHPUT SCREENING METHODOLOGY

(71) Applicant: The Regents of the University of California, Oakland, CA (US)

(72) Inventors: Alice SORAGNI, Los Angeles, CA (US); Peyton John TEBON, Los Angeles, CA (US); Luda LIN, Los Angeles, CA (US); Nasrin TAVANAIE, Los Angeles, CA (US); Bowen WANG, Los Angeles, CA (US)

(73) Assignee: The Regents of the University of California, Oakland, CA (US)

(21) Appl. No.: 18/037,646

(22) PCT Filed: Dec. 7, 2021
(86) PCT No.: PCT/US21/62264

§ 371 Date: May 18, 2023

Related U.S. Application Data
(60) Provisional application No. 63/122,258, filed on Dec. 7, 2020.

(51) Int. Cl. G01N 33/50; C12N 5/09

(52) U.S. Cl. CPC G01N 33/5047; G01N 33/5067; C12N 5/0693; C12N 2513/00; G01N 2800/52

(57) ABSTRACT

The present disclosure describes a high-throughput methodology for rapid and highly personalized screenings to identify efficacious anti-tumor immunotherapy regiments. In one embodiment, therapeutic agents or a combination thereof are screened by co-culturing tumor shaped organoid extrudates with a population of immune cells taken from the same patient. Reduced tumor cell functions or increased immune cell functions in the presence of the therapeutic agents or combination thereof would identify the therapeutic agents or combination thereof for treating the tumor in the patient.

### FIELD

The present disclosure relates in general to the field of biology, oncology and immuno-oncology. In one embodiment, the present disclosure provides methods for rapid bioprinting of cells and high-throughput screening of oncology or immuno-oncology therapies on shaped organoid extrudates.

### BACKGROUND

Immunotherapy for cancer, i.e., immuno-oncology (IO), is rapidly becoming the new cornerstone for cancer treatment alongside traditional surgery and medications. IO works by augmenting the immune system's natural ability to see and eliminate cancer cells much in the same way it protects us against infection from viruses and bacteria. As a living, dynamic system, the immune system is able to detect cancer anywhere in the body, which is especially important in treating patients with cancers that have spread or metastasized to other organs.

Immunotherapy is very different from other cancer treatments in terms of mechanism of action, response time, the potential for durable response, and side effects. IO therapy works with the immune system itself, unlike chemotherapy, which directly kills all rapidly dividing cells, including cancer and certain normal cells; or radiation, which targets and directly kills cancer cells and sometimes surrounding healthy cells; or small molecules that interfere with specific mechanisms required for cancer cells to grow. Immunotherapy has the potential to provide lasting protection against cancer after treatment (as the immune system has “memory”), lowering the risk of recurrence.

Recent clinical successes have resulted in Food and Drug Administration (FDA) approval of IO therapies, both alone and in combination with other treatments, for nearly 20 types of cancer, including advanced solid tumor and blood cancers. In bladder cancer, melanoma, and certain types of lung cancer, IO therapies have received FDA approval as first-line treatment, replacing or, in the case of combination approvals, improving conventional treatments like chemotherapy. IO therapies are also FDA-approved to treat some patients for whom prior treatments were ineffective, and clinical trials are ongoing to test the benefits of IO agents in many other types of cancer.

There are diverse IO drugs that target various steps in the anti-tumor immune response, including activation, elimination, and suppression. There are also different classes of IO with different mechanisms of action, including checkpoint blockade, cell therapy, monoclonal antibodies, vaccines, and immunomodulators like cytokines, pattern recognition receptors, and others. Different IO therapies have been shown to work differently in different cancers or subsets of cancers. For example, a certain type of CAR-T cell therapy has achieved an 83 percent response rate in B-cell acute lymphoblastic leukemia and a 50 percent response in diffuse large B cell lymphoma, but is not yet effective in treating solid tumors that arise in the lung, bladder, kidney, colon, brain, and other organs.

Whether a patient should receive immunotherapy with one drug or a combination of drugs with or without conventional therapy needs to be determined. It is believed that the key to determining which patients are likely to respond to IO treatment, or which treatments are more likely to work than others, is to consider the patient's unique situation such as their environment, lifestyle, treatment history, as well as the genetic makeup and gene expression of the patient's tumor.

Thus, there is a need to develop high-throughput screening methodology for rapid and highly personalized screenings to identify efficacious IO regiments.

### SUMMARY

In one embodiment, the present disclosure provides a method for identifying therapeutic agents or a combination thereof for treating a tumor in a patient, comprising the steps of (i) obtaining a sample of tumor cells optionally in single cell suspension, or as aggregates or clusters from the tumor of the patient; (ii) dispensing geometrically shaped organoid extrudates comprising the tumor cells into tissue culture wells of a tissue culture plate; (iii) co-culturing the shaped organoid extrudates with a population of assay cells in the tissue culture wells in the presence of a therapeutic agent or a combination thereof, wherein reduced tumor cell functions or increased assay cell functions in the presence of the therapeutic agent or combination thereof identifies the therapeutic agent or combination thereof for treating the tumor in the patient. In any embodiment, the assay cells may comprise immune cells or liver cells, i.e., hepatocytes.

In another embodiment, the present disclosure provides a method of treating a patient having a tumor, comprising the steps of (i) obtaining a sample of tumor cells optionally in single cell suspension, or as aggregates or clusters from the tumor of the patient; (ii) dispensing shaped organoid extrudates comprising the tumor cells into tissue culture wells of a tissue culture plate; (iii) co-culturing the shaped organoid extrudates with a population of assay cells in the tissue culture wells in the presence of a therapeutic agent or a combination thereof, wherein reduced tumor cell functions or increased assay cell functions in the presence of the therapeutic agent or combination thereof identifies the therapeutic agent or combination thereof for treating the tumor in the patient; and (iv) treating the patient with the therapeutic agent or combination thereof.

In another embodiment, the present disclosure provides an automated method for bioprinting cells or shaped organoid extrudates, comprising the steps of obtaining a tissue sample from a subject and preparing a single cell suspension of the tissue sample; mixing the single cell suspension, aggregates or clusters with a gel precursor solution, thereby forming a cell complex comprising cells and a matrix; dispensing the cell complex into one or more tissue culture wells by one or more dispensers of an automatic device, said dispensers have been calibrated so as to deposit the cell complex as a ring or square or other geometry around the perimeter of a base of the tissue culture wells.

In another embodiment, the present disclosure provides a method for screening candidate therapeutic agents for activities against tumor cells in the presence of an active cell complex comprising active cells that can metabolize the therapeutic agent or a combination thereof, the method comprising (i) a tissue culture plate comprising a plurality of tissue culture wells, the wells comprising shaped organoid extrudates, and (ii) the wells further comprising cells that may alter the sensitivity of the tumor to the agent in vivo, and provide an improved assessment of the potential benefit of the agent in the patient. In one embodiment, the active cell complex comprises liver cells or intestinal cells.

Thus, in one aspect, a method is provided for identifying therapeutic agents or a combination thereof for treating a tumor in a patient, comprising the steps of (i) obtaining a sample of tumor cells optionally in single cell suspension, or as aggregates or clusters from the tumor of the patient; (ii) extruding a collection of said tumor cells into tissue culture wells, such that the tumor cells form one or more shaped, three-dimensional organoid extrudates comprising said tumor cells; (iii) co-culturing said shaped organoid extrudate with a population of assay cells in said tissue culture wells in the presence of therapeutic agents or combination thereof, wherein reduced tumor cell functions or increased assay cell functions in the presence of said therapeutic agents or combination thereof identifies the therapeutic agents or combination thereof for treating the tumor in the patient. In one embodiment, the assay cells comprise immune cells, liver cells or intestinal cells.

Thus, In one aspect, a method is provided for treating a patient having a tumor, comprising the steps of: (i) obtaining a sample of tumor cells optionally in single cell suspension, or as aggregates or clusters from the tumor of the patient; (ii) extruding a collection of said tumor cells into tissue culture wells, such that the tumor cells form one or more shaped organoid extrudates comprising said tumor cells; (iii) co-culturing said shaped organoid extrudates with a population of assay cells in said tissue culture wells in the presence of therapeutic agents or combination thereof, wherein reduced tumor cell functions or increased assay cell functions in the presence of said therapeutic agents or combination thereof identifies the therapeutic agents or combination thereof for treating the tumor in the patient; and (iv) treating the patient with the therapeutic agents or combination thereof.

In some embodiments of the foregoing methods, the tumor cell functions comprise tumor cell growth, tumor cell viability or tumor cell mobility. In some embodiments, reduced tumor cell function is reduced mobility, reduced growth rate, or reduced cell viability. In some embodiments, reduced tumor cell function is tumor cell death. In some embodiments, reduced tumor cell functions and increased assay cell functions in the presence of said therapeutic agents or combination thereof identifies the therapeutic agents or combination thereof for treating the tumor in the patient.

In some embodiments, the assay cells are immune cells. In some embodiments, the immune cell functions comprise cytokine production or immune cell growth. In some embodiments, the assay cells are liver cells. In some embodiments, tissue culture plate is a 384-well, 96-well, 48-well, 24-well, 12-well or 6-well plate well. In some embodiments, the shaped organoid extrudates are deposited as rings around a perimeter at a bottom of said tissue culture wells. In some embodiments, the shaped organoid extrudates are deposited in a substantially square shape at the bottom of said tissue culture wells, and wherein the shaped organoid extrudates do not directly contact sidewalls of said tissue culture wells.

In some embodiments, the tumor cells are sarcoma cells. In some embodiments, the shaped organoid extrudates comprise a hydrogel. In some embodiments, the shaped organoid extrudates comprise alginate, collagen, gelatin or a basement membrane extract such as Matrigel®, Cultrex® BME, CELLINK GelXA, CELLINK LAMININK 111, or any combination thereof.

In some embodiments, the dispensing of the shaped organoid extrudates is performed manually or by automated bioprinting. In some embodiments, the dispensing of the shaped organoid extrudates is performed at a temperature of between about 0° C. and about 37° C. In some embodiments, the dispensing of the shaped organoid extrudates is performed at a temperature of about 37° C. In some embodiments, the dispensing of the shaped organoid extrudates is performed at a temperature of about 18° C. In some embodiments, the dispensing of the shaped organoid extrudates is performed through an orifice of about 0.1 to about 1 mm in diameter. In some embodiments, the dispensing of the shaped organoid extrudates is performed through an orifice of about 0.26 mm in diameter. In some embodiments, the dispensing of the shaped organoid extrudates is performed through an orifice of about 0.6 mm in diameter. In some embodiments, the tissue culture wells during said dispensing are maintained at a temperature of between about 0° C. to about 37° C. In some embodiments, the tissue culture wells during said dispensing are maintained at a temperature of about 37° C. In some embodiments, the dispensing of the shaped organoid extrudates is performed using automated bioprinting at an extrusion pressure of between about 1 kPa and about 150 kPa. In some embodiments, the dispensing of the shaped organoid extrudate is performed using automated bioprinting at an extrusion pressure of between about 10 and about 15 kPa. In some embodiments, the dispensing of the shaped organoid extrudates is performed using automated bioprinting at an extrusion pressure of about 15 kPa. In some embodiments, the dispensing of the shaped organoid extrudates is performed using automated bioprinting at a printhead speed of from about 1 to about 200 mm/second. In some embodiments, each shaped organoid extrudate comprises about 50 to about 5,000 cells per microliter. In some embodiments, each shaped organoid extrudate comprises about 500 to about 1,400 cells per microliter.

In some embodiments, the gel precursor solution comprises a hydrogel, the dispensing is at a temperature between about 0 and about 37° C., the print surface temperature is between about and about 37° C., the extrusion pressure is between about 1 and about 150 kPa, and the printhead speed is between about 1 and about 200 mm/s. In some embodiments, the gel precursor solution comprises a basement membrane extract, the dispensing is at a temperature between about 2 and about 37° C., the print surface temperature is between about 4 and about 37° C., the extrusion pressure is between about 1 and about 25 kPa, and the printhead speed is between about 5 and about 40 mm/s. In some embodiments, the gel precursor solution comprises a basement membrane extract, the dispensing is at a temperature between about 4 and about 18° C., the print surface temperature is about 17° C., the extrusion pressure is between about 1 and about 15 kPa, and the printhead speed is between about 10 and about 20 mm/s. In some embodiments, the gel precursor solution comprises CELLINK LAMININK, the dispensing is at a temperature between about 15 and about 25° C., the print surface temperature is between about 20 and about 25° C., the extrusion pressure is between about 35 and about 55 kPa, and the printhead speed is between about 10 and about 20 mm/s. In some embodiments, the gel precursor solution comprises PPO, the dispensing is at a temperature between about 20 and about 25° C., the print surface temperature is between about 20 and about 25° C., the extrusion pressure is between about 35 and about 150 kPa, and the printhead speed is between about 1 and about 80 mm/s.

In some embodiments, the tissue culture wells are in one or more 96-well plates, and each shaped organoid extrudate comprises about 50 to about 500 cells per microliter. In some embodiments, the tissue culture wells are in one or more 24-well plates, and each shaped organoid extrudate comprises about 500 to about 5,000 cells per microliter.

In some embodiments, the assay cells are obtained the said patient. In some embodiments, the assay cells are circulating immune cells or tumor infiltrating immune cells.

In some embodiments, the shaped organoid extrudates and the assay cells are submersed in a tissue culture medium in said tissue culture wells. In some embodiments, the shaped organoid extrudates and the assay cells are kept away from a central region of said tissue culture wells. In some embodiments, the assay cells comprise a hydrogel. In some embodiments, the hydrogel comprises alginate, collagen, gelatin or a basement membrane extract such as Matrigel®, Cultrex® BME, CELLINK GelXA, CELLINK LAMININK 111, or any combination thereof.

In some embodiments, the therapeutic agents comprise chemotherapeutic agents or immunotherapeutic agents.

In another aspect, an automated method is provided for bioprinting shaped organoid extrudates, comprising the steps of (i) obtaining a tissue sample from a subject and preparing a single cell suspension of said tissue sample; (ii) mixing said single cell suspension with a gel precursor solution, thereby forming a cell complex comprising cells and a matrix; (iii) dispensing said cell complex into one or more tissue culture wells by one or more dispensers of an automatic device, said dispensers have been calibrated so as to deposit said cell complex as a polygon or ring on a base of said tissue culture wells.

In some embodiments, the dispensing of the shaped organoid extrudates is performed at a temperature of between about 0° C. and about 37° C. In some embodiments, the dispensing of the shaped organoid extrudates is performed at a temperature of about 37° C. In some embodiments, the dispensing of the shaped organoid extrudates is performed at a temperature of about 18° C. In some embodiments, the dispensing of the shaped organoid extrudates is performed through an orifice of about 0.1 to about 1 mm in diameter. In some embodiments, the dispensing of the shaped organoid extrudates is performed through an orifice of about 0.26 mm in diameter. In some embodiments, the dispensing of the shaped organoid extrudates is performed through an orifice of about 0.6 mm in diameter. In some embodiments, the tissue culture wells during said dispensing are maintained at a temperature of between about 0° C. to about 37° C. In some embodiments, the tissue culture wells during said dispensing are maintained at a temperature of about 37° C. In some embodiments, the dispensing of the shaped organoid extrudates is performed using automated bioprinting at an extrusion pressure of between about 1 kPa and about 150 kPa. In some embodiments, the dispensing of the shaped organoid extrudate is performed using automated bioprinting at an extrusion pressure of between about 10 and about 15 kPa. In some embodiments, the dispensing of the shaped organoid extrudates is performed using automated bioprinting at an extrusion pressure of about 15 kPa. In some embodiments, the dispensing of the shaped organoid extrudates is performed using automated bioprinting at a printhead speed of from about 1 to about 200 mm/second. In some embodiments, each shaped organoid extrudate comprises about 50 to about 5,000 cells per microliter. In some embodiments, each shaped organoid extrudate comprises about 500 to about 1,400 cells per microliter. In some embodiments, a volume of cell suspension extruded per well in the 96-well plates is 10 microliters. In some embodiments, a volume of cell suspension extruded per well in the 96-well plates is 5 microliters. In some embodiments, a volume of cell suspension extruded per well in the 24-well plates is 70 microliters.

In some embodiments, the gel precursor solution comprises a hydrogel, the dispensing is at a temperature between about 0 and about 37° C., the print surface temperature is between about 0 and about 37° C., the extrusion pressure is between about 1 and about 150 kPa, and the printhead speed is between about 1 and about 200 mm/s. In some embodiments, the gel precursor solution comprises a basement membrane extract, the dispensing is at a temperature between about 2 and about 37° C., the print surface temperature is between about 4 and about 37° C., the extrusion pressure is between about 1 and about 25 kPa, and the printhead speed is between about 5 and about 40 mm/s. In some embodiments, the gel precursor solution comprises a basement membrane extract, the dispensing is at a temperature between about 4 and about 18° C., the print surface temperature is about 17° C., the extrusion pressure is between about 1 and about 15 kPa, and the printhead speed is between about 10 and about 20 mm/s. In some embodiments, the gel precursor solution comprises CELLINK LAMININK, the dispensing is at a temperature between about 15 and about 25° C., the print surface temperature is between about 20 and about 25° C., the extrusion pressure is between about 35 and about 55 kPa, and the printhead speed is between about 10 and about 20 mm/s. In some embodiments, the gel precursor solution comprises PPO, the dispensing is at a temperature between about 20 and about 25° C., the print surface temperature is between about 20 and about 25° C., the extrusion pressure is between about 35 and about 150 kPa, and the printhead speed is between about 1 and about 80 mm/s.

In some embodiments, the matrix of said cell complex is a hydrogel. In some embodiments, the gel precursor solution comprises alginate, collagen, gelatin or a basement membrane extract such as Matrigel®, Cultrex® BME, CELLINK GelXA, CELLINK LAMININK 111, or any combination thereof. In some embodiments, the gel precursor solution comprises a 3:4 ratio of MammoCult™ serum-free medium and either Matrigel® or Cultrex® BME.

In some embodiments, the tissue sample is a tumor sample. In some embodiments, the tumor sample is a sarcoma. In some embodiments, the single cell suspension, aggregates or clusters are obtained by enzymatic digestion. In some embodiments, the single cell suspension, aggregates or clusters is filtered through a 40 micrometer cell strainer. In some embodiments, the dispensers have been calibrated with an alignment guide inserted in a tissue culture well so as to position said dispensers at a desired position to dispense said cell complex.

In some embodiments, the variability, viability or integrity of the cell suspension is evaluated. In some embodiments, a coefficient of variation in number of cells dispensed into said tissue culture wells is within 5%. In some embodiments, cells dispensed into said tissue culture wells have a viability of at least 90%.

These and other aspects will be appreciated from the ensuing descriptions of the figures and detailed description.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

FIGS. 1A-1B present one embodiment of a high-throughput method for screening using organoids. FIG. 1A shows shaped organoid extrudates in a 96-well plate. Inset 1 is a top-view (left) and side-view (right) schematic of a Matrigel® ring with organoids in a well. The top-view corresponding photographed image is shown in panel 2, and a whole-well imaging using Celigo (Nexcelom) is shown in panel 3. While this figure depicts the shaped organoid extrudate positioned with its outer diameter adjacent to the interior perimeter of the well, in some embodiments the ring may be of a smaller outer diameter and not contact the wall of the well, or only contact it at one location if not centered in the bottom of the well. In other embodiments, the ring is replaced by a square or other geometry. FIG. 1B shows a schema of the screening protocol with representative bright field images. On Day 0, Matrigel® is seeded with tumor cells and rings are printed. On Day 1-3, organoid formation occurs within the rings. On Day 4-5, the tumor cells are exposed to therapeutic agent(s) being tested. On Day 6, the tumor cells are released and viability by ATP assay or other parameters are determined to assess the effect of the therapeutic agent(s). In other embodiments, cells are grown for different lengths of time (0-45 days), and exposed to therapy for different time durations (for example, about 12 hours to about 10 days).

FIGS. 2A-D show micrographs and data demonstrating how bioprinting of organoids enables efficient high-speed live cell interferometry (HSLCI). (A) Schematic of wells with mini-rings (top) and mini-squares (bottom) relative to HSLCI imaging path (dark arrows). The top views (left) demonstrate that transitioning from rings to squares increases the amount of material in the imaging path of the interferometer. The side views (right) show that organoids in the square geometry align to a single focal plane better than organoids in a ring. (B) Plasma treatment of the well plate prior to printing optimizes hydrogel construct geometry. Bioprinting Matrigel onto untreated glass (left) generates thick (˜200 μm) constructs that decreases the efficiency of organoid tracking by increasing the number of organoids out of the focal plane. Whole well plasma treatment (middle) increases the hydrophilicity of all well surfaces causing the Matrigel to spread thin (˜50 μm) over the surface; however, the increased hydrophilicity also draws bioink up the walls of the well. Plasma treatment with a well mask facilitates the selective treatment of a desired region of the well (right). This leads to optimal constructs with a uniform thickness of approximately across the imaging path. (C) Individual organoids can be tracked over time across imaging modalities. Five representative HSLCI images are traced to the imaging path across a brightfield image. (D) Cell viability of printed versus manually seeded MCF-7 cells in a Matrigel-based bioink. An ordinary one-way ANOVA was performed (p=0.0605) with post-hoc Bonferroni's multiple comparisons test used to compare all bioprinted conditions against the manually seeded control. Adjusted p-values were 0.0253, 0.6087, >0.9999, 0.1499 for print pressures 10, 15, 20, 25 kPa, respectively.

FIGS. 3A-3H show three embodiments of an alignment guide, which fit into a reference well in the microtiter plate, in order to align the needle for filling and subsequent automated steps for the other wells on the plate. FIG. 3A depicts a view of one type of alignment guide. FIG. 3B is a cutaway view showing a center channel into which a needle is positioned for alignment. FIG. 3C depicts another version of the alignment guide that fits into a reference well that provides alignment position for the needle. FIG. 3D shows that the interior channel is conical at the bottom to accommodate needles that may be bent from the manufacturer or during or between uses. The square channel offset from the center of the top face is for facile grabbing with forceps. FIG. 3E depicts another version of the alignment guide that fits into two reference wells that provides an alignment position for the needle with Z-height feedback. A lever 105 is incorporated in the design to provide an obvious indicator of proper alignment. When the needle passes through opening 101 (ensuring proper X and Y alignment) and presses on the reference position end of the lever 102, the flag end 103 rises through an opening in the surface as visual indicator of proper Z-alignment. The lever is used to translate a small change in Z-height of the reference position end to a larger, more obvious, change in the flag end. The conical opening 104 in the platform 106 prohibits the reference position end of the lever from rising above the height of the platform. FIGS. 3F-G depict section views of the aligner showing the lever in closer detail.

FIG. 4 depicts histology showing that bioprinting does not induce histological or morphological charges in organoids. H&E staining shows the development of multicellular organoids over time regardless of seeding method. The prevalence and size of multinuclear organoids increase with culture time. Ki-67/Caspase-3 staining demonstrates that most cells remain in a proliferative state throughout culture time. While apoptotic cells were observed in organoids cultured for 72 hours, the majority of cells show strong Ki-67 positivity. All images are 40× magnification and insets are 80× magnification. Ki-67 is stained brown, and caspase-3 is stained pink.

FIG. 5 depicts data demonstrating that bioprinting does not alter organoid transcriptomes. (A) Distributions of total number of transcripts detected (above) and transcript abundances (below) measured as transcripts per million (TPM) organized into groups of deciles based on median abundance. (B) RNA abundances (log 2 TPM) of manually seeded and bioprinted organoids at three different time points (t=1, 24, and 72 hours). Spearman's ρ was assessed for each association. We found strong associations between RNA abundances derived from printed and manually seeded organoids for both cell lines. (C) Volcano plots of Mann-Whitney U-test results for MCF7 and BT474 organoids with unadjusted p-values (left) and false discovery rate (FDR) adjusted p-values (right) comparing the RNA abundances of transcripts between manually seeded and printed tumor organoids. Fold change of RNA transcripts were assessed and log 2 transformed. No transcripts were preferentially expressed based upon seeding method for organoids of either cell line (n=0 out of 27,077 genes, q-value<0.1, Mann-Whitney U-test). (D) Median percent spliced in (PSI) of exon skipping isoforms were similarly distributed among MCF7 (left) and BT474 (right) derived organoids. Distribution of isoforms is consistent between manually seeded (blue) and bioprinted (red) organoids. PSI of 1 indicates that the isoform is exclusively an exon inclusion isoform, while a PSI of 0 indicates that the isoform is exclusively an exon skipping isoform.

FIG. 6 depicts a schema for bioprinting-based protocol for high-speed live cell interferometry. Extrusion-based bioprinting is used to deposit single-layer Matrigel constructs into a 96-well plate. Organoid growth can be monitored through brightfield imaging. After treatment, the well plate is transferred to the high-speed live cell interferometer for phase imaging. Coherent light illuminates the bioprinted construct and a phase image is obtained. Organoids are tracked up to three days using the interferometer and changes in organoid mass are measured to observe response to treatment.

FIG. 7 shows representative images of organoids treated with 10 μM staurosporine, 10 μM lapatinib, and 50 μM lapatinib. The white arrow annotates a BT-474 organoid that gains mass when treated with 10 μM lapatinib.

FIGS. 8A-8B depict the mass of tracked MCF-7, FIG. 8A, and BT-474, FIG. 8B, organoids by treatment. The left bars and pale points represent organoid mass after 6 hours of treatment and the right bars and dark points represent organoid mass after 48 hours of treatment.

FIG. 9 depicts scatterplots of normalized organoid pass over time. All organoid tracks for each treatment condition are shown on each plot. The mean normalized mass±standard deviation is also shown in orange (MCF-7) and blue (BT-474).

FIGS. 10A-10D depict hourly growth rate comparisons (percent mass change) between organoids treated with 10 μM staurosporine and vehicle, and 50 μM lapatinib and vehicle. FIG. 10A is MCF-7 cells treated with 10 μM staurosporine and vehicle; FIG. 10B is for MCF-7 cells treated with 50 μM lapatinib and vehicle; FIG. 10C is BT-474 cells treated with 10 μM staurosporine and vehicle; and FIG. 10D is for BT-474 cells treated with 50 μM lapatinib and vehicle.

FIGS. 11A-11B depict percent cell viability of MCF-7 cells, FIG. 11A, and BT-474 cells, FIG. 11B, in treated wells, determined by an ATP-release assay, where in each of FIGS. 11A-11B, p<0.05 is denoted by *, p<0.01 is denoted by **, and p<0.001 is denoted by ***.

FIG. 12 depicts immunohistochemistry staining of 3D cultures for HER2. BT-474 cells have amplified HER2 expression while MCF-7 cells express lower levels of HER2 and lack HER2 amplification.

FIG. 13 depicts estrogen receptor expression in BT-474 and MCF-7 organoids. Immunohistochemistry staining of 3D cultures for ER. Both BT-474 and MCF-7 cell lines are ER-positive.

FIGS. 14A-14B depict RNA fusions and editing sites. FIG. 14A depicts the number of RNA fusions detected by FusionCatcher by tumor organoid seeding method. The number of RNA fusions did not significantly differ between manually seeded and bioprinted organoids (pBT-474=0.179, pMCF-7=0.179). FIG. 14B depicts the number of adenosine-to-inosine (A-to-I) RNA editing sites detected by REDItools were not associated with tumor organoid development method (p=0.48, Mann-Whitney U-test). By cell line, the number of A-to-I RNA editing sites did not differ between printed and manually seeded organoids (pBT-474=0.1, pMCF-7=0.7).

FIGS. 15A-15B depict scatterplots and linear regression correlating initial organoid mass with specific organoid growth rate, for MCF-7 cells, FIG. 15A, and BT-474 cells, FIG. 15B. Specific growth rate (growth in mass as a percentage of total mass) versus initial organoid mass was plotted for all organoids tracked. Linear regression analysis showed a significant positive relationship between initial organoid mass and specific growth rate for MCF-7 organoids (95% confidence interval of slope is 0.1040 to 0.2993), FIG. 15A, but not for BT-474 organoids (95% CI of slope −0.041 to 0.2363), FIG. 15B.

FIGS. 16A-16B depict pie chart categorizations of organoid mass change for organoids treated with staurosporine, FIG. 16A, and organoids treated with lapatinib, FIG. 16B. The pie charts display the proportion of organoids that had gained mass (mass increase by >10%), remained stable (mass change <10%), or lost mass (mass loss of >10%) after 12 hours, 24 hours, and 48 hours.

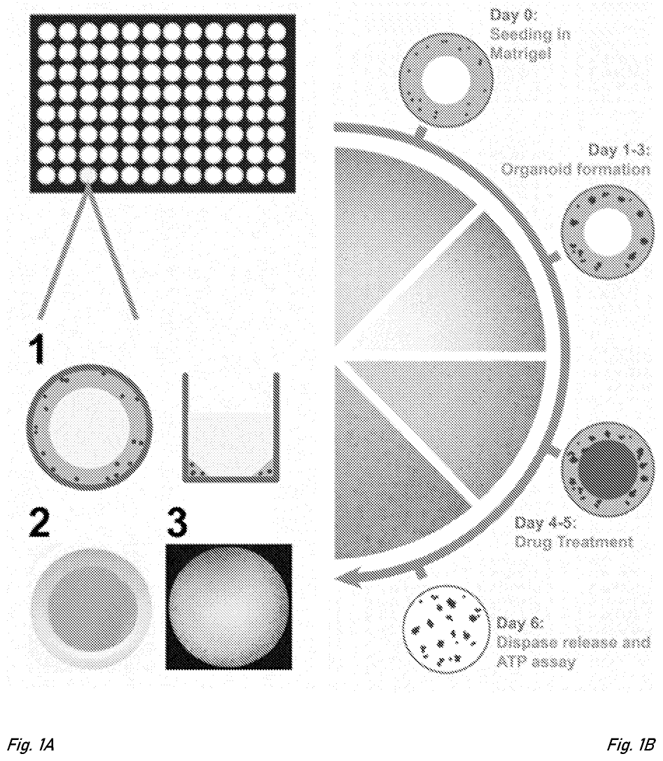

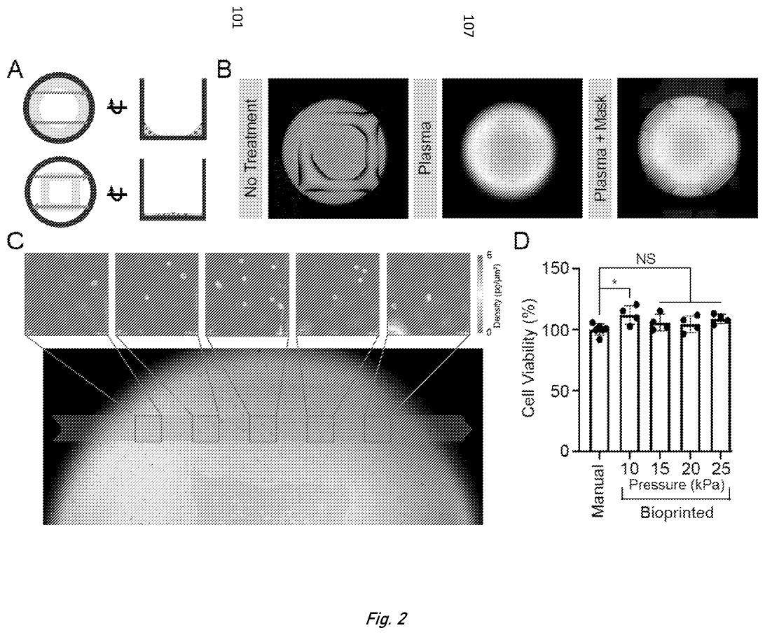

### DETAILED DESCRIPTION

In one aspect, the present disclosure provides a method for identifying therapeutic agents or a combination thereof for treating a tumor in a patient, comprising the steps of (i) obtaining a sample of tumor cells optionally in single cell suspension, or as aggregates or clusters from the tumor of the patient; (ii) dispensing shaped organoid extrudates comprising the tumor cells into tissue culture wells of a tissue culture plate; (iii) co-culturing the shaped organoid extrudates with a population of assay cells in the tissue culture wells in the presence of a therapeutic agent or a combination thereof, wherein reduced tumor cell functions or increased assay cell functions in the presence of the therapeutic agent or combination thereof identifies the therapeutic agent or combination thereof for treating the tumor in the patient. In one embodiment, the tumor cell functions comprise tumor cell growth or tumor cell mobility, or both. In any embodiment, “assay cells” may comprise immune cells or liver cells. In one embodiment, the assay cell functions comprise cytokine production or immune cell growth, or both. In some embodiments, assay cells are not included in the tissue culture wells. In some embodiments, assay cells are not included in the suspension. In some embodiments, assay cells are not included in the shaped organoid extrudate. In any embodiment, the assay cells may be immune cells or liver cells, i.e., hepatocytes.

In one embodiment, the tissue culture plate is a 384-well plate, 96-well plate, 24-well plate, 12-well plate or 6-well plate. In one embodiment, the tissue culture wells contain one or more shaped organoid extrudates comprising tumor cells, wherein each shaped organoid extrudate is deposited on the bottom of the tissue culture wells. For purposes of the present disclosure, a “shaped organoid extrudate” may comprise any 2-dimensional shape configuration, including, but not limited to sheets, rings, or polygons. A shaped organoid extrudate may be polygonal, annular, ovular, elliptical, toroid, lemniscate, X-shaped, C-shaped, etc., and is not limited to any particular 2-dimensional shape (except as context may otherwise dictate). Any type of tumor or cancer can be deposited at the bottom of the tissue culture wells. Examples of tumor or cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof. More than one shaped organoid extrudate may be deposited onto the bottom of a tissue culture well. For example, two organoids may be deposited beside each other in the same well. Concentric of square-shaped organoid extrudates may be deposited nested one in the other.

In one embodiment, the shaped organoid extrudate deposited at the bottom of the tissue culture wells comprises a hydrogel. For example, the shaped organoid extrudate comprises alginate, collagen, gelatin, or a basement membrane extract such as but not limited to Matrigel®, Cultrex® BME, CELLINK GelXA, CELLINK LAMININK 111, or any combination thereof. In one embodiment, the dispensing of the shaped organoid extrudates is independently performed by manual or automated bioprinting as disclosed herein. In one embodiment, the dispensing of the shaped organoid extrudates is performed at a temperature of between about 0° C. and about 37° C. In one embodiment, the dispensing of the shaped organoid extrudates is performed at a temperature of about 37° C. In one embodiment, the dispensing of the shaped organoid extrudates is performed through an orifice or needle of about 0.1 to about 1 mm in diameter. In one embodiment, the dispensing of the shaped organoid extrudates is performed through an orifice or needle of about 0.26 mm in diameter. In one embodiment, the tissue culture wells during the dispensing of cells are maintained at a temperature of between about 0° C. to about 37° C. In one embodiment, the tissue culture wells during the dispensing of cells are maintained at a temperature of about 37° C. In one embodiment, the temperature is between about 2° C. and about 37° C. In one embodiment, the dispensing of the shaped organoid extrudates is performed by automated bioprinting at an extrusion pressure of between about 1 kPa and about 150 kPa. In one embodiment, the dispensing of the shaped organoid extrudates is performed by automated bioprinting at an extrusion pressure of between about 10 kPa and about 15 kPa. In one embodiment, the dispensing of the shaped organoid extrudates is performed by automated bioprinting at an extrusion pressure of about 15 kPa. In one embodiment, the dispensing of the shaped organoid extrudates is performed by automated bioprinting at a printhead speed of from about 1 to about 200 mm/second. In one embodiment, the shaped organoid extrudate comprises about 50 to about 5,000 cells per microliter. In one embodiment, the shaped organoid extrudate comprises about 500 to about 1,400 cells per microliter. In one embodiment, the tissue culture wells are in one or more 96-well plates, and the shaped organoid extrudate comprises about 50 to about 500 cells per microliter. In another embodiment, the tissue culture wells are in one or more 24-well plates, and the shaped organoid extrudate comprises about 500 to about 5,000 cells per microliter.

In one embodiment, the assay cells used in the above method are obtained from the patient that has the tumor. In another embodiment, the assay cells are normal cells of any origin. In another embodiment, the assay cells are immune cells, which may be circulating immune cells or tumor infiltrating immune cells. In one embodiment, the immune cells are seeded in suspension. In another embodiment, the assay cells are seeded interspersed in the same hydrogel matrix as the tumor organoids. In another embodiment, assay cells are seeded in another bioprinted concentric hydrogel ring.

In one embodiment, the shaped organoid extrudates and optionally the assay cells are submerged in a tissue culture medium in the tissue culture wells. In one embodiment, the shaped organoid extrudates and the assay cells are kept away from a central region of the tissue culture wells. In one embodiment, the assay cells comprise a hydrogel. In one embodiment, the hydrogel comprises alginate, collagen, gelatin, a basement membrane extract such as but not limited to Matrigel®, Cultrex® BME, CELLINK GelXA, CELLINK LAMININK 111, or any combination thereof. Such hydrogels and other materials or combinations for automated or manual printing are also referred to as bioinks.

In one embodiment, the therapeutic agents screened in the above method comprise one or more chemotherapeutic or targeted agents or one or more immunotherapeutic agents or any combination thereof.

In another embodiment, the present disclosure provides a method for treating a patient having a tumor, comprising the steps of (i) obtaining a sample of tumor cells from the tumor of the patient; (ii) dispensing shaped organoid extrudates comprising the tumor cells into tissue culture wells of a tissue culture plate; (iii) co-culturing the shaped organoid extrudates with a population of assay cells in the tissue culture wells in the presence of a therapeutic agent or a combination thereof, wherein reduced tumor cell functions or increased assay cell functions in the presence of the therapeutic agent or combination thereof identifies the therapeutic agent or combination thereof for treating the tumor in the patient; and (iv) treating the patient with the therapeutic agent or combination thereof. In one embodiment, the tumor cell functions comprise tumor cell growth or tumor cell mobility, or both. In any embodiment, assay cells may be immune cells or liver cells. In one embodiment, the immune cell functions comprise cytokine production or immune cell growth, or both. In some embodiments, assay cells are not included. In any embodiment, reduced tumor cell function may refer to reduced cell motility, cell mobility, cell growth, or cell viability. In any embodiment, reduced tumor cell function may refer to tumor cell death.

In another embodiment, the present disclosure provides an automated method for bioprinting shaped organoid extrudates, comprising the steps of (i) obtaining a tissue sample from a subject and preparing a suspension of single cell or clusters of the tissue sample; (ii) mixing the single cell suspension, aggregates or clusters with a gel precursor solution, thereby forming a cell complex comprising cells and a matrix; (iii) dispensing the cell complex into one or more tissue culture wells by one or more dispensers of an automatic device, the dispensers have been calibrated so as to deposit the cell complex as a ring or other geometrical shape around the base of the tissue culture wells. In one embodiment, the dispensing of the cell complex is performed at a temperature of between about 0° C. and about 37° C. In one embodiment, the dispensing of the cell complex is performed at a temperature of about 37° C. In one embodiment, the dispensing of the cell complex is performed through an orifice of about 0.1 to about 1 mm in diameter. In one embodiment, the dispensing of the cell complex is performed through an orifice of about 0.26 mm in diameter. In one embodiment, prior to dispensing, the cell complex is retained in a reservoir maintained at a temperature of between about 0° C. to about 37° C. In one embodiment, prior to dispensing, the cell complex is retained in a reservoir maintained at a temperature of about 37° C. In one embodiment, the tissue culture wells during dispensing are maintained at a temperature of between about 0° C. to about 37° C. In one embodiment, the tissue culture wells during dispensing are maintained at a temperature of about 37° C. In one embodiment, the dispensing of the cell complex is performed at an extrusion pressure of between about 1 and about 150 kPa. In one embodiment, the dispensing of the cell complex is performed at an extrusion pressure of between about 12 and about 15 kPa. In one embodiment, the dispensing of the cell complex is performed at an extrusion pressure of about 15 kPa. In one embodiment, the dispensing of the cell complex is performed at a printhead speed of from about 1 to about 200 mm/second. In one embodiment, the cell complex comprises about 50 to about 5,000 cells per microliter. In one embodiment, the cell complex comprises about 500 to about 1,400 cells per microliter. In one embodiment, the tissue culture wells are in one or more of 96-well plates, and the cell complex comprises about 50 to about 500 cells per microliter. In another embodiment, the tissue culture wells are in one or more of 24-well plates, and the cell complex comprises about 500 to about 5000 cells per microliter.

In some embodiments, the shaped organoid extrudate is printed around the perimeter of the bottom of the well. In some embodiments, the shaped organoid extrudate is smaller than the diameter of the interior of the well. For example, a 3.3 mm diameter ring may be printed at the bottom of wells that are 6.35 mm in diameter. In some embodiments, an orifice or needle of 0.4 to 1.0 mm in diameter is used to print a ring of a width approximately between 0.4 and 1.0 mm. A shaped organoid extrudate of a particular diameter represents the mean of the inner and the outer diameters. In one embodiment, a volume of 10 microliters of cell suspension is extruded per well in the 96-well plates. In another embodiment, a volume of 70 microliters of cell suspension is extruded per well in the 24-well plates. In one embodiment, the matrix of the cell complex is a hydrogel, for example, the gel precursor solution comprises Matrigel®, Cultrex® BME, CELLINK GelXA, CELLINK LAMININK 111, or any combination thereof. In one embodiment, the gel precursor solution comprises a 3:4 ratio of MammoCult™ serum-free medium and either Matrigel® or Cultrex® BME.

In one embodiment of the above automated bioprinting method, the tissue sample is a tumor sample. Examples of types of tumors have been described above. In one embodiment, the single cell suspension, aggregates or clusters of tumor cells is obtained by enzymatic digestion of a tumor sample, such as obtained by biopsy or surgery. In one embodiment, the single cell suspension, cell aggregate or cell cluster is filtered through a 40-micrometer cell strainer.

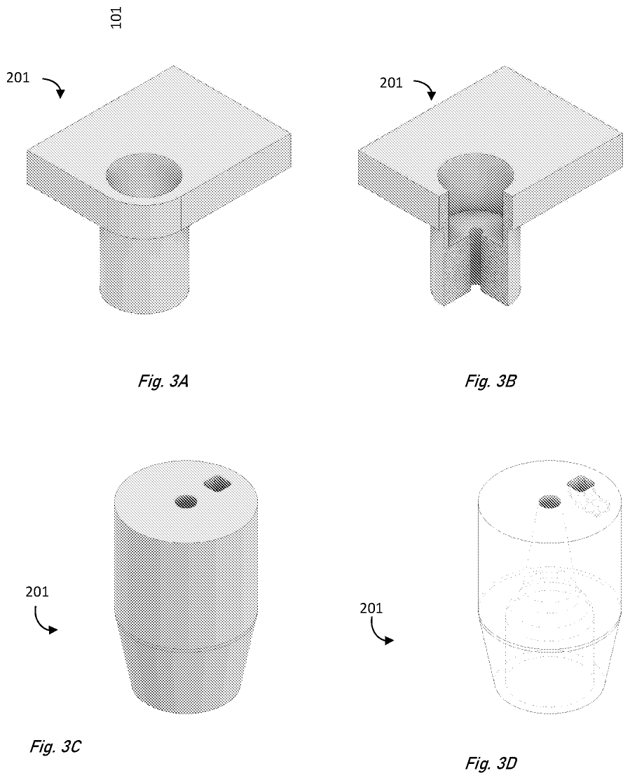

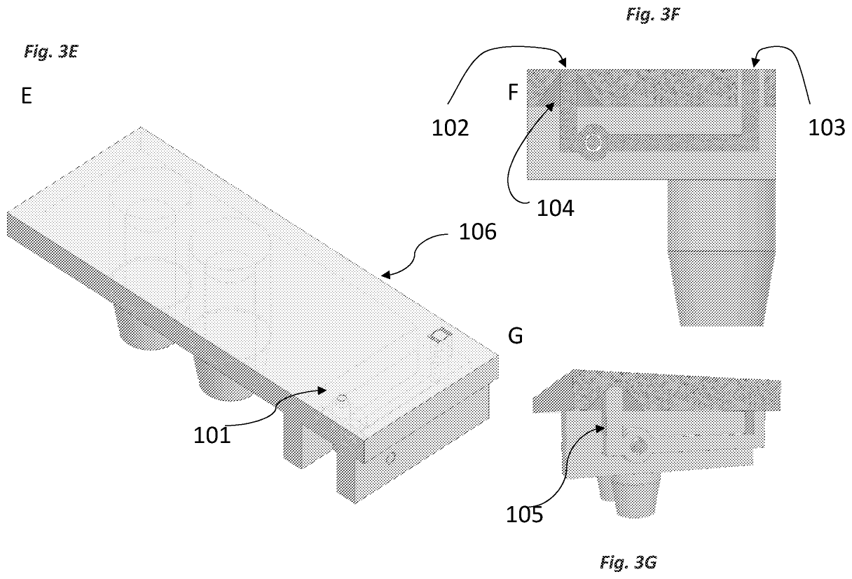

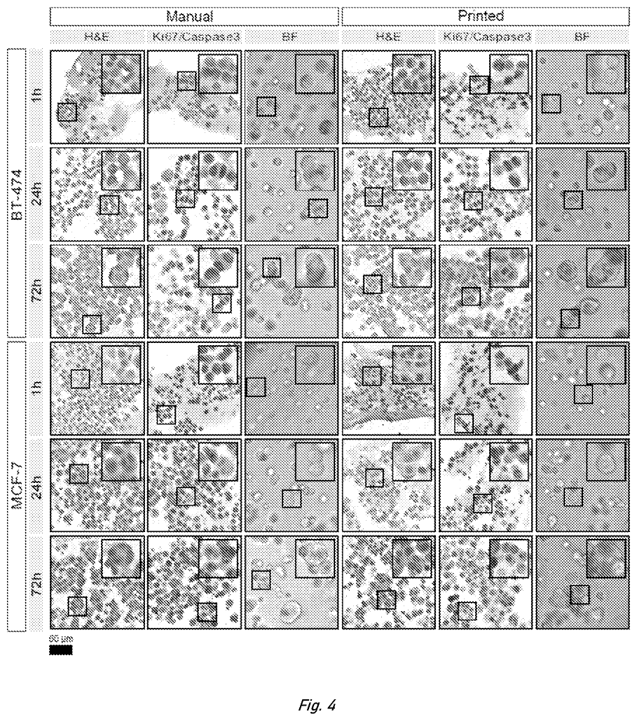

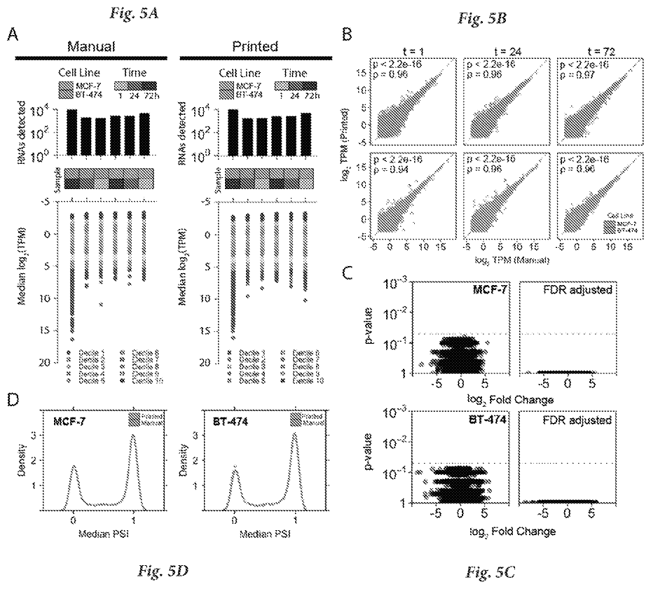

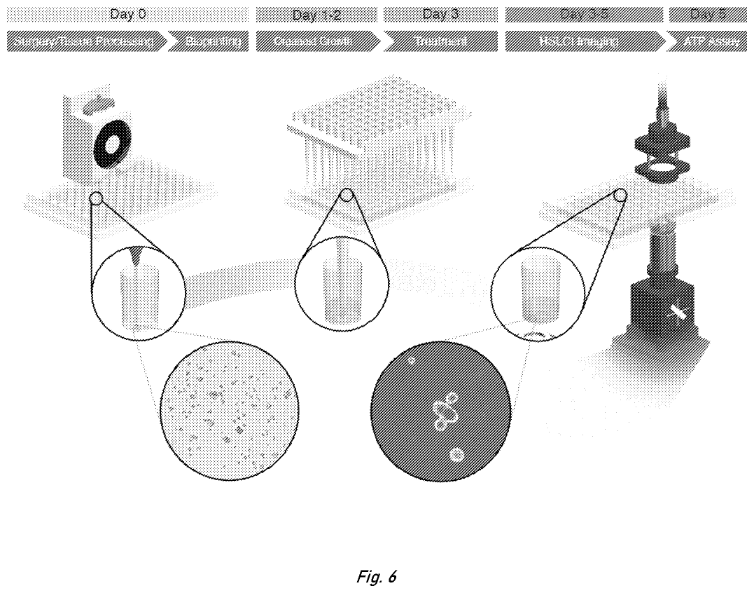

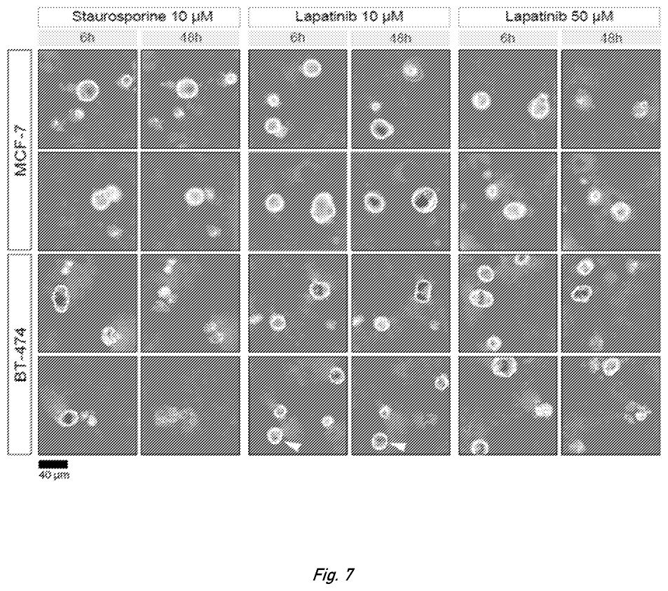

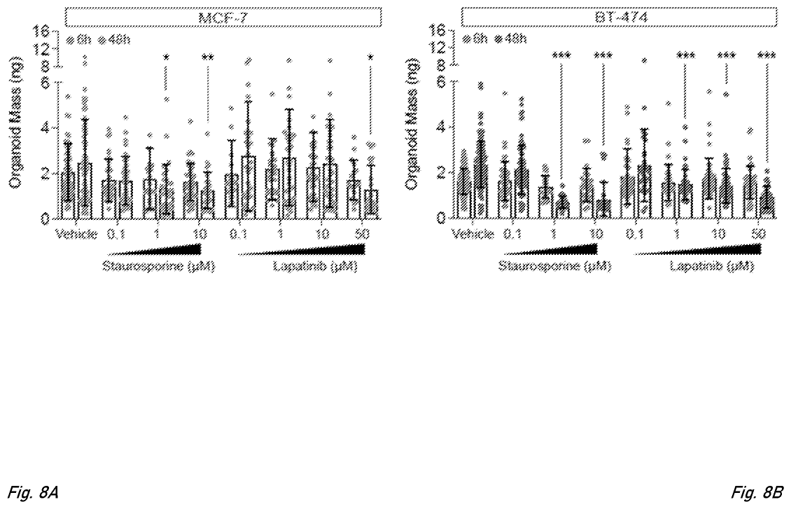

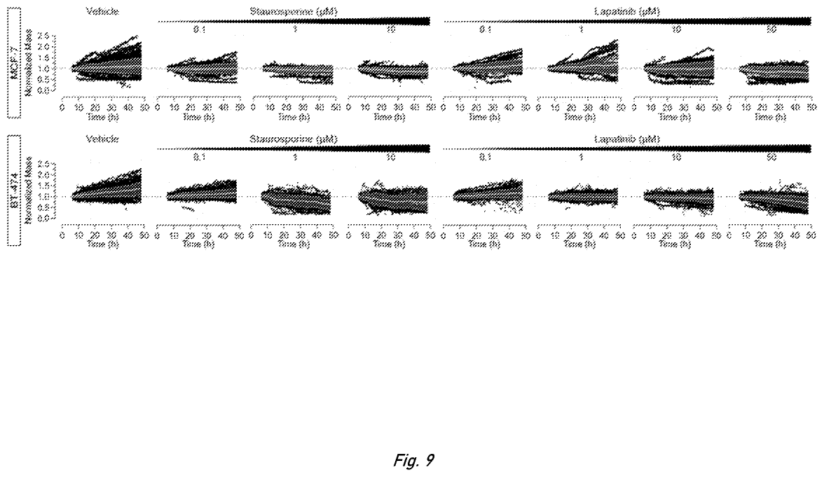

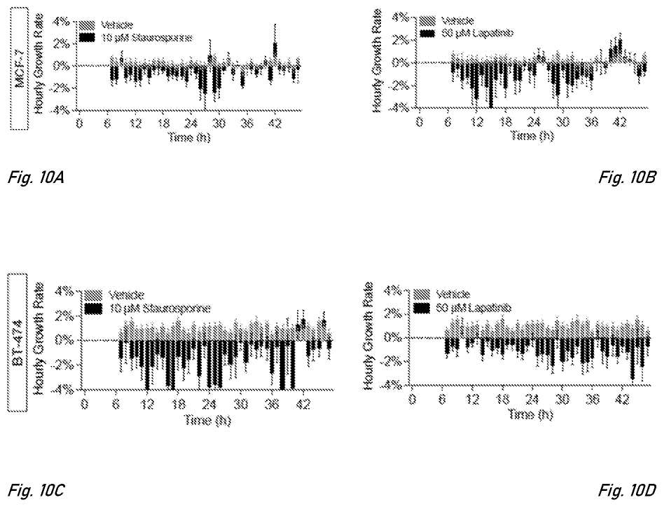

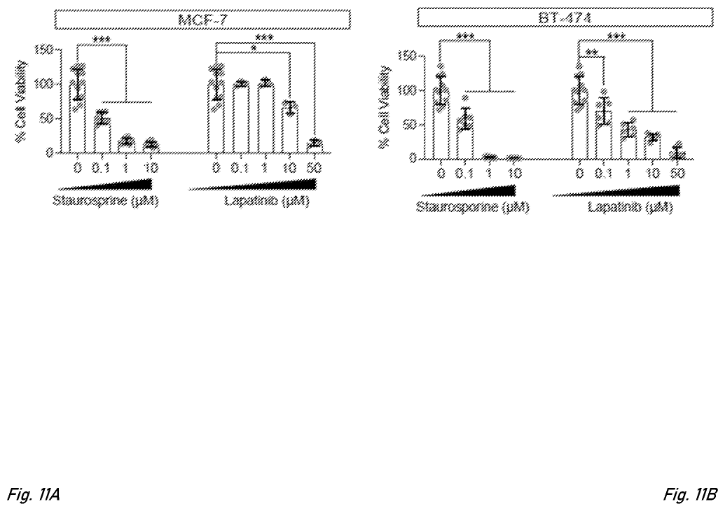

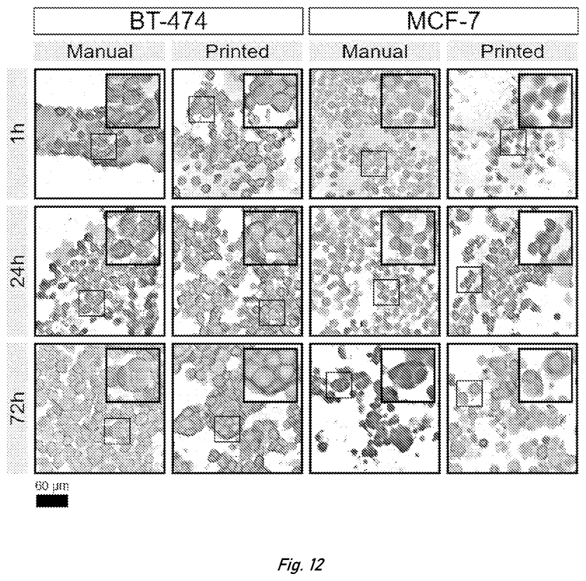

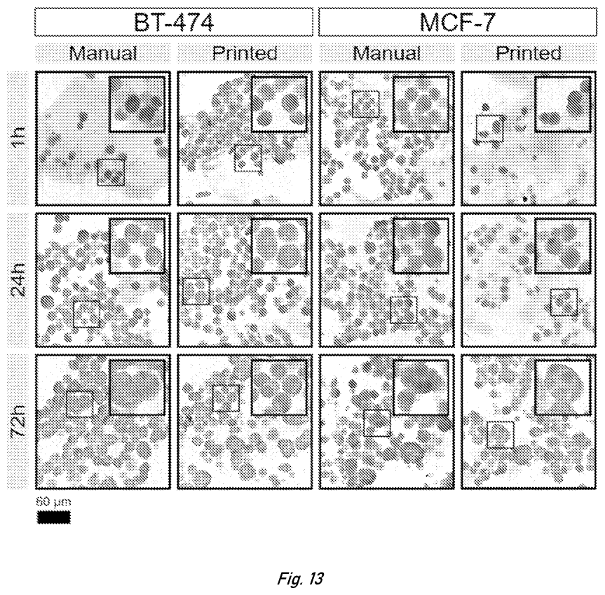

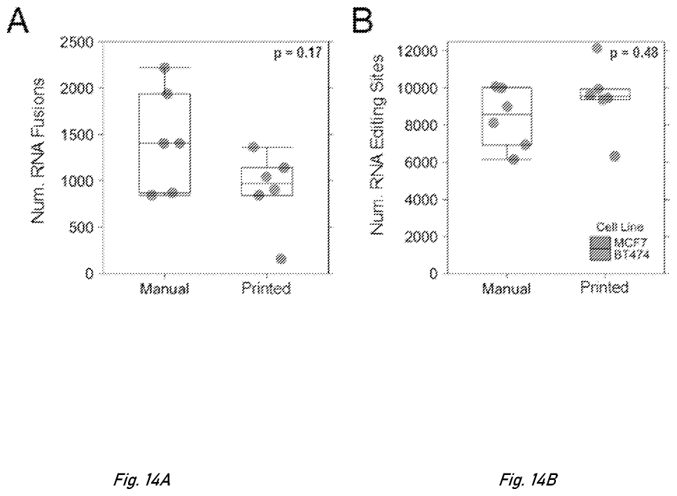

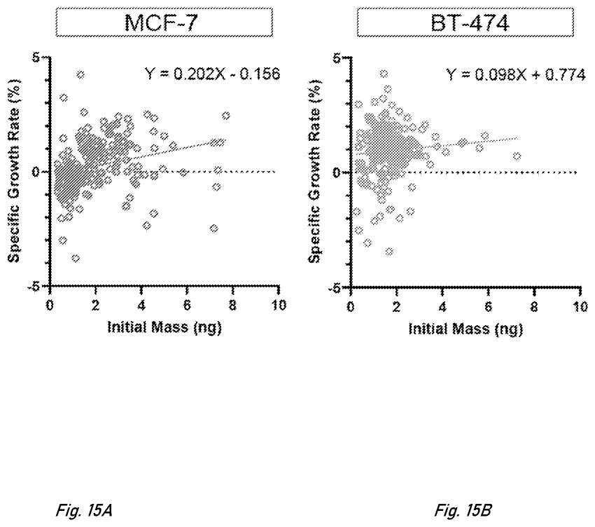

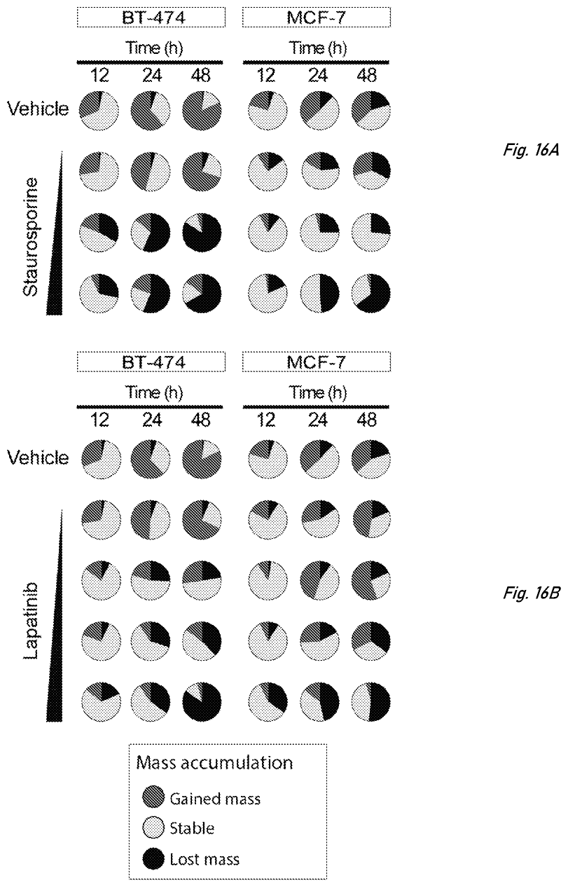

In one embodiment of the above automated bioprinting method, the dispensers (e.g., orifice or needle) have been calibrated with an alignment guide 201 inserted in one tissue culture well on the plate, so as to position the dispensers at a desired position to dispense the cell complex to form the shaped organoid extrudates. Another version of the alignment guide is shown in FIGS. 3E-G that fits into two reference wells that provides an alignment position for the needle with Z-height feedback. A lever 105 is incorporated in the design to provide an obvious indicator of proper alignment. When the needle passes through opening 101 (ensuring proper X and Y alignment) and presses on the reference position end of the lever 102, the flag end 103 rises through an opening in the surface as visual indicator of proper Z-alignment. The lever is used to translate a small change in Z-height of the reference position end to a larger, more obvious, change in the flag end. The conical opening 104 in the platform 106 prohibits the reference position end of the lever from rising above the height of the platform. FIGS. 3F-G depict section views of the aligner showing the lever in closer detail. The aligner device and use are an aspect of the disclosure herein.

As used herein, the term shaped organoid extrudate or the term organoid ring may be used to refer to the printed ring or any other polygonal shape comprising matrix and cells, before the cells therein have formed organoids. In one embodiment, the variability, viability, biological properties or integrity of the cell suspension is evaluated. Variability may be assessed using a variety of assays that quantify live cell number, such as metabolic assays, assays for live/dead staining, or organoid counting/area calculations performed through image analysis. Viability can similarly be assessed using a metabolic assay; in such an example, in one embodiment, several (e.g., four) rings will be plated by hand and used as viability benchmarks. Using a luminescence-based assay, the percent viability will be calculated by dividing the luminescent signal from wells with bioprinted rings by the luminescent signal of the manually seeded controls. Biological properties may be characterized by transcriptomics, genomics or metabolomics analyses. Ring integrity may be qualitatively assessed using high-content brightfield imaging in which observations can be made regarding cracks, material deformation, or missing segments and quantified by machine learning approaches. In one embodiment, the coefficient of variation in number of cells dispensed into the tissue culture wells is within 5-25%. In one embodiment, the variation of cells dispensed is within 5-20%. In one embodiment, the variation of cells dispensed is within 5-15%. In one embodiment, the variation of cells dispensed is within 5-10%. In one embodiment, the variation of cells dispensed is less than 5%. In another embodiment, the cells dispensed into the tissue culture wells have a viability of at least 80% with respect to the viability in the original cell suspension. In another embodiment, the cells dispensed into the tissue culture wells have a viability of at least 85% with respect to the viability in the original cell suspension. In another embodiment, the cells dispensed into the tissue culture wells have a viability of at least 90% with respect to the viability in the original cell suspension. In another embodiment, the cells dispensed into the tissue culture wells have a viability of at least 95% with respect to the viability in the original cell suspension.

In one embodiment, multiple hydrogel structures can be deposited in a single well. These structures can vary in size, shape, material composition, and cell content. Additional structures may be deposited by independent print heads or a single printhead. All subsequent analysis remains identical for multi-part constructs. In one embodiment, the coefficient of variation in number of cells dispensed into the tissue culture wells is within 5-25%. In another embodiment, the cells dispensed into the tissue culture wells have a viability of at least 80% with respect to the original cell suspension.

In some embodiments of the above automated printing method, the shapes of the shaped organoid extrudates deposited onto the bottom of the wells of a tissue culture plate are closed polygons. In some embodiments of the above automated printing method, the shapes of the shaped organoid extrudates deposited onto the bottom of the wells of a tissue culture plate are open polygons or non-polygons. In some embodiments, the shapes of the shaped organoid extrudates are ellipses. As used herein, “shaped organoid extrudate” includes any closed polygonal or non-polygonal two-dimensional shape that fits within the confines of the bottom of a tissue culture well. In some embodiments, shaped organoid extrudates may be printed in the shape of a circle, an oval, a square, a rectangle, or another closed polygonal or non-polygonal two-dimensional shape. The shape of the shaped organoid extrudate is not limiting. In some embodiments, the geometry of the shaped organoid extrudates deposited onto the bottom of the wells of a tissue culture plate varies among different wells of the plate. In some embodiments, the volume of the shaped organoid extrudates deposited onto the bottom of the wells of a tissue culture plate varies among different wells of the plate. In some embodiments, some wells of a tissue culture plate will not contain shaped organoid extrudates.

As used herein, the singular form “a”, “an” and “the” include plural references unless the context clearly dictates otherwise.

Throughout this application, various embodiments of the present disclosure may be presented in a range format. It should be understood that the description in range format is merely for convenience and brevity and should not be construed as an inflexible limitation on the scope disclosed herein. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps and variations thereof are described. This description is not intended to be limiting and changes in the components, sequence of steps, and other variations would be understood to be within the scope of the present disclosure.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable subcombination or as suitable in any other described embodiment. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the present disclosure.

### Example 1

Bioprinting Organoids for High-Throughput Drug Screening and Therapy Selection

Currently, a protocol for screening therapies on patient-derived organoids relies upon manual deposition of the cell-laden gel around the perimeter of a well. This procedure is laborious and requires a refined technique thus significantly limits both the number of patients that can be tested as well as the number of drugs to be screened for each patient. Additionally, subsequent imaging and analysis of the organoids is complicated by inconsistencies in the focal planes of interest and meniscus effects generated by the well geometry.

To overcome the above challenges, the present example describes an automated organoid seeding process utilizing bioprinting to improve efficiency and consistency while also making data collection and analysis easier and more robust.

Bioprinting will be used to generate consistent, robust hydrogel constructs. In one embodiment, the bioprinting approach can be optimized by using sarcoma cell lines selected for their ability to generate tumor organoids in vitro. The cells can be printed directly into 96-well plates using a CELLINK BioX. The performance of bioprinted mini-rings can be compared to manually pipetted mini-rings plated as described previously. The rings can be evaluated based on three major criteria: variability, integrity, and cell viability. Variation in size, shape, and cell number must be minimized between samples. Additionally, the gel structures printed must withstand agitation and shear associated with plate handling and automated fluid exchanges. In one embodiment, matrices composed of a 3:4 ratio of MammoCult™ serum-free medium and either Matrigel® or Cultrex® BME can be used. However, these materials have temperature-dependent viscosities that pose unique challenges during printing. They can be tested and optimized by controlling printhead and deposition surface temperature. These challenges are addressed by controlling the ambient temperature in the room and using refrigerated rockers/shakers for precise environmental temperature control. Moreover, other commercially available matrices (e.g., CELLINK GelXA, CELLINK LAMININK 111, GelXA CELLINK LAMININK+, Bioink) and simple gel-forming solutions (e.g., gelatin, collagen, laminin) can also be used. For each material, resultant cell viability and proliferation can be analyzed with Calcein AM and propidium iodide staining and an ATP assay, respectively. Conditions that yield viable organoids in consistent, stable gels can be validated with patient-derived cells.

In one embodiment, the optimized bioprinting protocol can be tested on organoids derived from different patient samples. In addition to the criteria outlined above, the printing protocol for patient samples must also preserve the native phenotype of the tumor. This can be evaluated by immunohistochemistry (IHC) and hematoxylin and eosin (H&E) staining of paraffin-embedded samples to qualitatively characterize cell morphology and phenotypic markers. RNA sequencing (RNAseq) can also be conducted to observe gene expression. The histology and gene expression profile of the organoids can be compared against the original sample to ensure that cell behavior is not altered by bioprinting.

The tumor organoids made by bioprinting can be implemented in the drug screening process for patients. These patients can be selected based on the available amount of tissue, as concurrent screening using existing methods can also be conducted for comparison. In one embodiment, the bioprinting process includes printing 5000 cells/well embedded within the optimized hydrogel matrix in 96-well plates. The organoids can be incubated for 72 hours with bright field images taken every 24 hours. After the incubation period, the liquid in the wells are removed and replaced with fresh medium containing the drug of interest. All drugs can be tested with a minimum of 4 replicates and at concentrations of, for example, 0.1, 1, and 10 μM. Treatment is repeated twice over consecutive days. In one embodiment, a drug array containing nearly 500 compounds including standard chemotherapeutics and targeted therapies (e.g., kinase, PARP, proteasome, and HDAC inhibitors) can be used. After treatment, an ATP assay (CellTiter-Glo 3D, Promega) on each sample is performed to measure sensitivity. Sensitivity is calculated by comparing the viability of the organoids in each well. Viability is determined by comparing the number of live cells in a given well against the untreated negative control wells. The ATP assay is a surrogate for estimating the number of live cells by yielding a quantifiable luminescence directly proportional to the number of live cells. The sensitivity of the bioprinted organoids can be compared against those produced by the current manual bioprinting protocol to ensure equivalence.

In one embodiment, the bioprinting conditions can also be optimized using patient-derived cells. Materials and printing parameters may be varied. For example, the ATP assay is a surrogate for viability and biased toward drugs affecting metabolic pathways. Thus, a label-free image-based approach can be developed to quantify drug effects using morphological analysis including analysis over periods of time. Similar methods have been developed for the characterization of single cells and organoids. Standard image analysis based on binary masking and morphological characterization can be performed using MATLAB or Python and parameters such as area, circularity, and optical density can be correlated with viability to identify significant metrics. A neural network can be trained to predict the viability of the organoids by training the model on bright field images and extracted feature characteristics. This will leverage the existing database of sarcoma organoid images labeled with their sensitivity to therapy (viability after treatment). The overall goal of this analytical tool is to eliminate the need to perform a disruptive chemical assay to evaluate the effects of drugs on the organoids.

Thus, the results of the analysis of the effect of the therapeutic agent or a combination thereof, and optionally including an immunotherapy agent or combination thereof, on the activity of function of the tumor cells, and/or the activity or function of assay cells, are used to identify a potential therapeutically effective therapeutic regimen for treating the patient from whom the tumor cells were obtained. In some embodiments, the patient is administered a chemotherapy agent or combination identified using the methods described herein as effective in reducing tumor activity or function. In some embodiments, the patient is administered an immunotherapy agent or combination identified by the methods described herein as effective in increasing activity of function of assay cells, and may also have an effect on reducing tumor cell activity or function. In some embodiments, the patient is administered a chemotherapy agent or combination identified using the methods described herein as effective in reducing tumor activity or function. In some embodiments, the patient is administered a chemotherapeutic agent or combination thereof, and an immunotherapy agent or combination thereof, the combination of chemotherapeutic agent(s) and immunotherapy agent(s) identified by the methods described herein as effective in increasing activity of function of assay cells, effective in reducing tumor cell activity or function, or both.

### General Procedures

In one embodiment, the bioprinting process starts with harvesting patient-derived tissue samples that are dissociated into a single cell suspension, aggregates or clusters upon mincing and treatment with collagenase IV. The cell suspension is then filtered through a 40 μm cell strainer. The cell suspension is mixed into a gel precursor solution and deposited in a ring (or other) shape around the bottom of individual wells automatically using a bioprinter. In some embodiments, the ring is deposited around the perimeter of the bottoms of the wells. Once the solution has gelled in a warm environment, medium is added. After 48-72 hours, the medium is replaced with medium containing the drugs of interest for screening. Images of each well are taken every 24 hours until the end of the experiment. Viability is tracked using a chemical assay and supplemented with image analysis. All media exchanges are managed by an automated fluid handler and imaging can also be managed by an automated imaging system.

Thus, and as described in further examples herein, a patient's tumor sample can be evaluated for sensitivity to a large number of therapeutic and chemotherapeutic agents to identify an optimal therapeutic regimen for the patient. And also as described herein, the inclusion within the shaped organoid extrudate or medium is a sample of (non-tumor) assay cells, e.g., liver or immune cells, that may modulate the activity of the tested therapeutic agents on the patient's tumor, an improved selection of potentially efficacious treatment regimen may be identified for that particular patient's tumor or for that therapeutic regimen for other patients.

Guidance as to each of the steps the printing process, and preparation therefor, is provided below, which guidance is merely exemplary and non-limiting, and may be modified as taught by the disclosure herein to achieve the desired results.

### Printer Preparation

(1) Turn printer on.
(2) Set the temperature of the bed, printhead(s), and incubator(s) to the desired temperatures.
(3) Load the pre-specified printing protocol (e.g., STL or Gcode file) into the machine.
(4) Prepare cell suspensions in the desired complex of bioink gel and media.
(5) Agitate cell-loaded bioink(s) at the printing temperature for 30 minutes to ensure homogeneous cell dispersion and temperature-equilibration.
(6) Load the cell-loaded bioink(s) in the printer-specific bioink reservoir(s).
(7) Agitate excess bioink reservoirs at the printing temperature until use.
(8) Insert the bioink reservoir(s) into the printer. Attach the desired print nozzle or needle to the printer.
(9) Prime the nozzle or needle to ensure that it is filled with the bioink and ready to deposit material.
(10) Calibrate the X, Y, and Z positions to the center and bottom of the calibration well for the printer. See the Printer Alignment protocol below for more detail. The protocol described below calibrates to well H1; however, this is an arbitrary selection.
(11) Once the machine is calibrated, run the printing protocol.
(12) After the machine has finished printing, collect the plate for downstream analysis.

### Printhead Alignment

In one embodiment, the absolute coordinates of the printer are set to recognize the center of the reference well as (0, 0, 0) (X, Y, Z). In one embodiment, the alignment process can be performed as follows:
(1) Place well plate on the print surface;
(2) Insert 3D printed alignment guide (see FIGS. 3A-3D) in well H1;
(3) Align X and Y to the center of the alignment guide in H1;
(4) Move the printer needle to the proper Z height by slowly approaching the surface of the well until the tip of the needle has made contact with the plastic of the plate;
(5) Save these coordinates as (0, 0, 0).
(6) Repeat steps 3-5 for multiple print heads, if necessary.

### Printer Requirements

Temperature control over bioink reservoir: 0-37° C.;
Temperature control over substrate: 0-37° C.;
Lateral (XY) resolution: 50 μm;
Vertical (Z) resolution: 50 μm;
Printer speed: 0-100 mm/s;
Bioink orifice diameter (needle size): 0.4-1 mm.

MATERIAL REQUIREMENTS

| | Material | | Print Surface | | Extrusion | | Printhead | |
|---|---|---|---|---|---|---|---|---|
| Material | Temperature | | Temperature | | Pressure | | speed | |
| Hydrogels | 0-37° | C. | 0-37° | C. | 1-150 | kPa | 1-200 | mm/s |
| Basement | 2-37° | C. | 4-37° | C. | 1-20 | kPa | 5-40 | mm/s |
| membrane | | | | | | | | |
| extract | | | | | | | | |
| working | | | | | | | | |
| range | | | | | | | | |
| Basement | 4-18° | C. | 17° | C. | 1-15 | kPa | 10-20 | mm/s |
| membrane | | | | | | | | |
| extract | | | | | | | | |
| optimal | | | | | | | | |
| range | | | | | | | | |
| CELLINK | 15-25° | C. | 20-25° | C. | 35-55 | kPa | 10-20 | mm/s |
| LAMININK | | | | | | | | |
| PPO | 20-25° | C. | 20-25° | C. | 30-150 | kPa | 1-80 | mm/s |

The foregoing table provides guidance on selecting the parameters for bioprinting shaped organoid extrudates as disclosed herein for some examples of hydrogels. Variations with the selections is with the teachings herein.

In some embodiments, Matrigel® and BME are printed between 4° C.-18° C. at pressures between 1-20 kPa with the print surface at 17° C. and the printhead speed between 10 and 20 mm/s.

In one embodiment, the term “hydrogels” encompasses both commercially available (e.g., CELLINK Series, Allevi Liver dECM) and lab-developed materials (e.g., GelMA, ColMA) derived from natural (e.g., collagen, gelatin, alginate) or synthetic biocompatible polymers or poloxamers (e.g., Pluronic, PEG, PPO). These materials are referred to herein as bioink. In some embodiments, the hydrogel comprises a basement membrane extract.

In one embodiment, the Matrigel®-based bioink is a complex of Matrigel® and cell culture medium. Several ratios can be used when mixing a variety of gel matrices (e.g., Matrigel®, BME, etc.) with cell culture media (e.g., MammoCult™, RPMI, DMEM). For example, 3 parts of medium is used to 4 parts of Matrigel. In other embodiments, the ratio of medium to Matrigel® can be 1:2, 1:1, 2:1, or 3:1, or pure Matrigel® solutions can be used. In other embodiments, a thickening agent such as xanthan gum or a cellulose derivative such as carboxymethylcellulose may be included at from about 1% to about 20% to modify the mechanical properties.

### Cell Seeding Requirements

In one embodiment, cells are seeded in a range of 500-25,000 cells per well in a 96-well plate or 10,000-200,000 cells per well in a 24-well plate. This is derived from bioink solutions seeded at a density of 50 cells/μl to 5000 cells/μl. Cell density is dependent on a range of factors. Cells can be tumor cells or normal cells of any type.

### Shape Requirements

The printed shape can be any closed or non-closed polygonal or non-polygonal shape composed of a single or multiple layers. Multiple shapes can be printed within the same well either side-by-side, concentrically or overlaid, by way of non-limiting examples. Each shape/structure can contain singular, multiple, or no cell types. Shapes must be able to be circumscribed by a circle with the internal diameter of the given well plate and must not occupy the central region of the well, for example, within a 0.5 mm radius of the center. In one embodiment, culture wells with a 6.35 mm diameter base can be used and any printing shapes can be used provided that the outer edge of the printing needle remains within the 6.35 mm circular boundary and outside of the excluded central radius. In one embodiment, a ring is printed having a mean diameter of 3.3 mm, and a width of 1 mm. As printed in the center of the well, the printed ring does not contact any wall of the well.

### Example 2

High-Throughput Drug Screening Method

Tumor organoids are capable of reproducing many important features of the cancer they are generated from, including heterogeneity, cell organization, and drug response. The present example presents a robust automated high-throughput screening platform that takes advantage of a unique geometry to test the responses of patient-derived tumor organoids to hundreds of therapeutic agents. Screening results can become available within a week from surgery, a timeline compatible with therapeutic decision-making.

The shaped organoid extrudate referred to elsewhere herein is referred to in this example as a ring shape, a mini-ring, a maxi-ring, a mini-square, tumor organoid, among other related descriptions referring to the shaped organoid extrudate, typically containing tumor cells, as disclosed herein throughout.

In one embodiment, the screening process starts with harvesting patient-derived tumor tissue samples that are dissociated into a single cell suspension, aggregates or clusters upon mincing and treatment with enzymes such as collagenase IV. The cell suspension is then filtered through a cell strainer, e.g., a 40 μm cell strainer. The cell suspension is mixed with a gel precursor solution and deposited in a ring shape around the bottom of individual tissue culture well manually using a pipette or automatically using a bioprinter as described herein. Once the solution has gelled in a warm environment, culture medium is added. After a desired period of time, e.g., 48 hours, the culture medium is replaced with medium containing the drugs of interest for screening. Images of each well can be taken at desirable time intervals, e.g., every 24 hours, until the end of the screening. In one embodiment, cell viability can be tracked using a chemical assay and supplemented with image analysis. All media exchanges can be managed by an automated fluid handler, and imaging can also be managed by an automated imaging system.

### Methods.

### 2D Cell Culture.

MCF-7 and BT-474 breast adenocarcinoma cell lines were obtained from the American Type Culture Collection (ATCC). All cell lines were grown for a maximum of 10 passages in RPMI 1640 (Gibco 22400-089) supplemented with 10% fetal bovine serum (FBS, Gibco 16140-071) and 1% antibiotic-antimycotic (Gibco 15240-062). Both cell lines were authenticated by short tandem repeat profiling using the GenePrint 10 kit (Laragen).

### Manually Seeded 3D Organoids.

Organoids were seeded manually according to published protocols. Briefly, single cells suspended in a 3:4 mixture of MammoCult™ (StemCell Technologies 05620) and Matrigel® (Corning 354234) were deposited around the perimeter of the wells of either 24-well or 96-well plates. The cell suspension was kept on ice throughout the seeding process to prevent gelation of the Matrigel. To seed organoids in a 96-well plate (Corning 3603), a pipette was used to distribute 5 μL of cell suspension (5×105 cells/mL) along the bottom perimeter of each well; this “mini-ring” seeding geometry facilitated automatic changes of media and addition of drugs with a liquid handling system. Every eight wells, the cell suspension was briefly vortexed, and the pipette tip was exchanged. Once all mini-rings are generated, plates were incubated at 37° C. and 5% CO₂ for 20 minutes to solidify the Matrigel®, and 100 μL of pre-warmed MammoCult™ was added to the center of each well using an epMotion 96 liquid handler (Eppendorf). To generate larger rings (maxi-rings) in 24-well plates (Corning 3527), 70 μL of cell suspension (1.4×106 cells/mL) was deposited around the perimeter of each well. After every three wells, the cell suspension was vortexed, and the pipette tip was replaced. Following seeding, the plate was incubated at 37° C. and 5% CO₂ for 45 minutes to solidify the Matrigel®, and 1 mL of pre-warmed MammoCult™ was added to the center of each well.

### 3D Printing Plasma Masks.

Custom well masks were designed to meet the specifications of the well plates that were used in these experiments. The design was generated in Inventor 2020 (Autodesk) and printed using a Form3B (FormLabs). We elected to use the Biomed Amber resin (FormLabs) to generate these constructs due to its biocompatibility and ability to be autoclaved. The design was exported as an STL file and imported into the PreForm (FormLabs) software to arrange the parts. After printing, parts were post-processed in two washes of isopropanol, air-dried for at least 30 minutes, and cured for an additional 30 minutes at 70° C. in the Form Cure (FormLabs).

### Bioprinted 3D Organoids.

Cells were bioprinted using a CELLINK BioX with a Temperature-Controlled Printhead. Gcode files were written to print the desired single-layer geometry. We wrote standardized blocks of Gcode encode the print path for the repeated geometries. MATLAB (MathWorks, Inc.) was used to integrate these standardized blocks into full Gcode files with the defined coordinates for each well. We used 8-well plates when printing the larger constructs as the depth of the well in a standard 24-well plate prohibited the use of 0.5″ length needles. Large rings were necessary to deposit a sufficient number of cells for RNA-Seq and IHC. Four rings with a diameter of 14.5 mm were printed for RNA sequencing (approximately 200,000 cells total), while four sets of concentric 14.5 mm, 12.5 mm, and 10.5 mm diameter rings were used for IHC analysis (approximately 500,000 cells total). We printed mini-squares with side length 3.9 mm for drug screening and HSLCI imaging. The mini-squares were inscribed within the circular well with sides parallel to the sides of the well plate. The open center of the constructs facilitates automatic manipulation with fluid handling equipment while the sides of the square are positioned to maximize the number of organoids imaged by HSLCI. The bioprinting process utilized the same material deposited for manually seeded organoids. A single cell suspension, aggregates or clusters in a 3:4 mixture of MammoCult™ and Matrigel® was prepared on ice. After vortexing briefly to homogenize, the mixture was transferred into a 3 mL syringe by removing the plunger and capping the opposite end. Once the plunger was replaced, the syringe was inverted, and bubbles were forced out of the tip. The material was then transferred to a room temperature 3 mL bioprinter cartridge (CELLINK) by connecting the syringe and cartridge with a double-sided female Luer lock adapter (CELLINK). Any air bubbles in the syringe were removed and the loaded cartridge was incubated in a rotating incubator (Enviro-Genie, Scientific Industries) for 30 minutes at the print temperature.

During the incubation period, the printer was sterilized with the built-in UV irradiation function and the printhead was set to the print temperature. During this time, we treated the 96-well plates with oxygen plasma to improve the hydrophilicity of the surface. The well masks were autoclaved prior to use. The masks were inserted into the well plate and pressed in contact with the glass surface. Rubber bands were used to hold the masks in place and ensure conformal contact was maintained throughout the plasma treatment. The well plates were treated with oxygen plasma in a PE-25 (Plasma Etch) for 30-90 seconds, 15 minutes prior to bioprinting. After plasma treatment, the well plate was placed in the bioprinter and Automatic Bed Levelling (ABL) was performed.

Once the incubation period ended, we attached a 0.5″ 25-gauge needle and loaded the cartridge into the pre-cooled printhead. We primed the needle by extruding a small volume of material at 15 kPa prior to calibrating the printer. Just before printing, we cleared the needle of the gelled material by extruding a small volume using 40 kPa prior to starting the print. To create constructs of the appropriate thicknesses, prints in 8-well plates were extruded at 15 kPa while prints in 96-well plates were extruded at 12-15 kPa. The bioprinter completes the deposition process for 96-well plates in approximately four minutes. After printing, the constructs were incubated at 37° C. for at least 30 minutes to solidify the matrix and 100 μL of MammoCult™ medium was then added.

### Assessment of Viability.

The viability of manually seeded and bioprinted organoids was compared using an ATP detection assay. Manually seeded organoids were prepared in accordance with the protocol described above and previously published in Phan, N. et al. A simple high-throughput approach identifies actionable drug sensitivities in patient-derived tumor organoids. Commun. Biol. 2, 1-11 (2019); Nguyen, H. T. L. & Soragni, A. Patient-Derived Tumor Organoid Rings for Histologic Characterization and High-Throughput Screening. STAR Protoc. 1, (2020), doi.org/10.1016/j.xpro.2020.100056; and Shihabi, A. A. et al. Personalized chordoma organoids for drug discovery studies. bioRxiv 2021.05.27.446040 (2021) doi:10.1101/2021.05.27.446040, which are each incorporated by reference herein. To assess the viability of bioprinted organoids, we prepared the bioink and bioprinter as described. Instead of printing the bioink into a well plate, we extruded 100 μL, of bioink into and Eppendorf tube for each print pressure (10, 15, 20, and 25 kPa). We seeded four 10 μL rings in a 96-well plate using the extruded bioink. We then added 50 μL of 5 mg/mL Dispase (Life Technologies 17105-041) solution to each well and incubated for 25 minutes. After shaking for 5 minutes on an orbital shaker at 80 RPM, we added 75 μL of CellTiter-Glo® Luminescent Cell Viability Reagent (Promega G968B) to each well and followed the manufacturer's instructions, which are incorporated by reference herein. Luminescent readings were taken on a SpectraMax iD3 (Molecular Devices) plate reader. The viability of each well was calculated by normalizing the luminescent signal to the average signal from the manually seeded control wells. An ordinary one-way ANOVA with post-hoc Bonferroni's multiple comparisons test was performed in GraphPad Prism.

### Immunohistochemistry.

Immunohistochemical staining was performed on manually seeded and bioprinted organoids seeded in 24-well plates or 6-well plates, respectively. A detailed procedure has been published independently in Nguyen, H. T. L. & Soragni, A. supra. The samples were prepared for histological analysis by carefully aspirating all media from the well without disrupting the construct and washing with pre-warmed phosphate buffer solution (PBS). The PBS was added dropwise to the center of the well to avoid delamination and fragmentation. After aspirating all remaining liquid from the well, we added 10% buffered formalin (VWR 89370-094) and followed by incubating at 37° C. After the 5-minute incubation, the plates were transferred to ice for 30 minutes before storage in a 4° C. refrigerator until collection. The fixed organoids were harvested within 3 days using a pipette tip to scrape the surface of the wells, the organoids were subsequently transferred to a conical tube. The organoids were pelleted by centrifugation at 2000×g for 5 minutes and the supernatant was aspirated; this process was repeated twice to remove as much liquid as possible. HistoGel (Thermo Scientific HG-40000-012) was then added to the pellet. Cells were mixed with the HistoGel by briefly vortexing before placing on ice to solidify. Cassettes were labeled and 5 μL of HistoGel was used to coat a region of the bottom surface. Once solidified, the cell pellet in HistoGel was placed in the cassette and an additional 4 μL of HistoGel was added to the top of the pellet for stability. We wrapped cassettes in parafilm and chilled them on ice for 3 minutes before unwrapping and immersing in 70% ethanol. The cassettes were then sent to the UCLA Translational Pathology Core Laboratory (TPCL) for dehydration and paraffin embedding. After embedding, 8 μm thin sections were cut from the paraffin block.

Slides were baked for 20 minutes at 45° C. and de-paraffinized in xylene followed by washes in ethanol and deionized water. For H&E staining, a Hematoxylin and Eosin Stain Kit (Vector Labs H-3502) was used according to the manufacturer's protocol, which is incorporated by reference herein. For Ki-67/Caspase-3, HER2, and ER staining, Peroxidazed-1 (Biocare Medical PX968M) was applied for 5 minutes at room temperature to block endogenous peroxidases. Next, antigen retrieval was performed by immersing slides in Diva Decloaker (Biocare Medical DV2004LX) using an NxGEN Decloaking Chamber (Biocare Medical) to heat to 110° C. for 15 minutes. An additional 2-minute peroxidase blocking step was implemented after antigen retrieval in the Ki-67/Caspase-3 protocol. Blocking was performed at RT for 5 minutes with Background Punisher (Biocare Medical BP947H). Primary Ki-67/Caspase-3 staining was performed overnight with pre-diluted Ki-67/Caspase-3 (Biocare Medical PPM240DSAA) solution at 4° C., and secondary staining was performed with Mach 2 Double Stain 2 (Biocare) solution for 40 minutes at room temperature. Primary antibodies for HER2 (Novus Biologicals, CL0269) and ER (Abcam, E115) staining were diluted 1:100 in Da Vinci Green Diluent (Biocare Medical PD900L). The HER2 antibody was incubated overnight at 4° C. while the ER antibody was incubated at room temperature for 30 minutes. Secondary staining was performed with Mach 3 Mouse Probe and Mach 3 Mouse HRP-Polymer for HER2 and Mach 3 Rabbit Probe and Mach 3 Rabbit HRP-Polymer for ER, all secondary staining steps were 10 minutes. Chromogen development was performed with Betazoid DAB (Biocare Medical, BDB2004) and the reaction was quenched with deionized water. Counterstaining was performed with 20% Hematoxylin (Thermo Scientific #7221) for 7.5 minutes. Slides were dehydrated in a sequence of ethanol and xylene baths before cover slips were applied with Permount (Fisher Scientific SP15-100). Imaging was performed with a Revolve microscope (Echo Laboratories). White balancing of the images was performed in Adobe Photoshop.

### Sample Preparation for RNA Sequencing.

Organoids were released from the Matrigel® in preparation for whole transcriptome sequencing (RNA-Seq). After aspirating the media from each ring, 1 ml of cold Dispase was added per ring. After a 20-minute incubation at 37° C., the cell suspension was collected and pelleted by centrifugation at 1500×g for 5 minutes and washed with 45 ml of PBS before centrifuging again at 2000×g for an additional 5 minutes. Once all liquid was aspirated, the tubes were rapidly frozen and stored at −80° C. Frozen cell pellets (approximately 200,000 cells) were then transferred to the Technology Center for Genomics & Bioinformatics (TCGB) at UCLA for RNA sequencing. Sequencing was performed in one lane of the NovaSeq SP (Illumina) using the 2×150 bp paired-end protocol.

### RNA Sequencing Data Processing and Analysis.

FASTQ files were processed using the UCLA-CDS pipeline that includes pipeline-align-RNA v6.2.2, pipeline-quantitate-RNA v2.0.1, pipeline-quantitate-SpliceIsoform v2.0.6, pipeline-call-RNAEditingSite v5.0.0, pipeline-call-FusionTranscript v1.1.0. Pipeline-align-RNA v6.2.2 used a combination of FASTQC v0.11.9, fastp v0.20.1, STAR v2.7.6a, HISAT2 v2.2.1, Pipelinequantitate-RNA used kallisto v0.46.0, samtools v1.10, rsem 1.3.3. Pipeline-quantitate-SpliceIsoforms used rmats v4.1.0. Pipeline-call-RNAEditingSite used REDItools2 v1.0.0. Pipeline-call-FusionTranscripts used STAR-Fusion v1.9.1, fusioncatcher v1.33, Arriba v2.1.0. Samples with low transcript abundance (TPM<0.1; transcripts per million) were excluded resulting in 27,077/67,060 transcripts included in the analysis. We excluded splice isoforms with missing data in 5 or more samples (8,561 out of 17,449) due to low power. RNA editing sites were filtered to include adenosine to inosine events with sufficient coverage (q30>10) and frequencies above 0.9. Poly-A depleted RNA included annotated microRNAs (miRNA), while poly-A enriched RNA included coding mRNAs. Raw and processed data will be made available in GEO.

We used a Mann-Whitney U-test to compare the distributions of RNA abundances, number of transcript fusions, and editing sites between bioprinted and manually seeded tumor organoids. We adjusted for multiple comparison using the false discovery rate (FDR) method. FDR values (q<0.1) was the criteria for strong associations. Statistical analyses and data visualization were performed in the R statistical environment (v4.0.2) using the BPG (v6.0.1) package.

### High-Speed Live Cell Interferometer.

HSLCI has been described previously in Huang, D. et al. High-Speed Live-Cell Interferometry: A New Method for Quantifying Tumor Drug Resistance and Heterogeneity. Anal. Chem. 90, 3299-3306 (2018); and Murray, G. F. et al. Live Cell Mass Accumulation Measurement Non-Invasively Predicts Carboplatin Sensitivity in Triple-Negative Breast Cancer Patient-Derived Xenografts. ACS Omega 3, 17687-17692 (2018). The HSLCI platform is a custom-built inverted optical microscope coupled to an off-axis quadriwave lateral shearing interferometry (QWLSI) camera (SID4BIO, Phasics, Inc.), motorized stages (Thorlabs) holding a single, standard-footprint (128×85 mm), glass-bottom multi-well plate, and a piezo-actuated dynamic focus stabilization system that enables continuous and repeated image collection over many fields of view (FOVs) within the sample area. All of the platform's hardware and software components are available commercially.

The HSLCI platform was installed inside a standard cell culture incubator. For all growth kinetics studies and drug screening, organoids were imaged in 96-well glass-bottom plates (Cellvis P96-1.5H-N) using a 40× objective (Nikon, NA 0.75). Organoids were seeded as single-cell suspensions in a 3:4 mixture of MammoCult™ and Matrigel® as described above and were grown at 37° C. and 5% CO₂ in MammoCult™ medium supplemented with 1% antibiotic-antimycotic (Gibco 15240-062). Plates were wrapped in with parafilm to limit evaporation and placed in the interferometer. The typical imaging interval was 10 minutes between successive frames at the same FOV.

### Drug Screening.

All drug treatments of 3D organoids were performed in serum-free conditions in MammoCult™ medium supplemented with 1% antibiotic-antimycotic (Gibco 15240-062). A detailed protocol for the drug screening has been published previously in Phan, N. et al. supra; and Nguyen, H. T. L. & Soragni, supra. Briefly, the culture medium was fully removed three days after seeding and replaced with 100 μL of MammoCult™ medium containing the indicated treatments using an automated pipetting system (EpMotion® 96). After treatment, we transferred the organoids to the HSLCI for imaging. Organoids were imaged by HSLCI between 6 hours and 48 hours after treatment. After imaging, we performed an ATP assay to assess cell viability in accordance with the manufacturer's instructions. The media was aspirated from each well and replaced with 50 μL of 5 mg/mL Dispase (Thermo-Fisher) solution to digest the Matrigel. After a 25-minute incubation at 37° C., the plate was placed on an orbital shaker for 5 minutes at 80 RPM. We then added 30 μL of CellTiter-Glo® reagent (Promega), sealed the plate with film, covered the plate with foil to protect from light, shook the plate for 5 minutes at 80 RPM, and incubated at room temperature for an additional 20 minutes. We used a SpectraMax iD3 plate reader to measure luminescence. The program parameters for luminescence readings were 5 minutes of shaking prior to reading, reading all wavelengths, and integrating signal over 500 ms. Organoid viability within each well was calculated by dividing the luminescent signal from each well by the mean luminescence of the control (1% DMSO) wells. Two-tailed independent t-tests were performed to assess the statistical significance of the differences in organoid mass and cell viability. P-values less than 0.05 were deemed significant.

### HSLCI Data Analysis.

Image processing and data analysis were performed on a downstream computer using a custom, multi-step MATLAB pipeline. First, interferograms captured by the SID4Bio QWLSI camera were converted to phase shift images using the SID4 software development kit for MATLAB (Phasics). Next, phase images are segmented into individual cells or organoids using a combination of a Gaussian lowpass filter and a watershed transform. Mass is extracted from the segmented area of each object by integrating the phase shift over that area and then multiplying by the experimentally determined specific refractive increment of 1.8×10−4 m³/kg. Finally, objects identified by image segmentation were tracked over time using a particle tracking code originally developed by John Crocker and David Grier for IDL, and subsequently adapted for MATLAB by Daniel Blair and Eric Dufresne. See Crocker, J. C. & Grier, D. G. Methods of Digital Video Microscopy for Colloidal Studies. J. Colloid Interface Sci. 179, 298-310 (1996), incorporated by reference herein.

To ensure quality of hourly growth rates recorded, data were filtered such that only biomass tracks with a 75th percentile of mass of 350 pg and only segments of biomass tracks (mass vs. time) exhibiting sufficiently low local variability were included. The minimum mass filter ensures that our data does not include organoids that are already dead at the start of tracking. Variability was assessed by calculating the standard deviation of normalized mass changes within a bin of 11 mass versus time data points. The maximum allowed standard deviations were 2.8% and 3.6% for MCF-7 and BT-474, respectively. This accounts for the noise introduced by cell debris or out-of-focus objects and excludes portions of tracks if they are interrupted by debris or move out of focus. Furthermore, segments of mass versus time tracks with high local variability were fit to a sigmoidal filter and those with a goodness-of-fit better than a user-defined threshold were kept, to include tracks corresponding to cells that start alive and in focus and die over the duration of tracking.

### Results.

Bioprinting enables seeding cells in Matrigel® in conformations that are suitable for imaging applications. To address current limitations and facilitate non-invasive, label-free, real time imaging of 3D organoids by HSLCI, we optimized an automated cell printing protocol using an extrusion bioprinter. Our original platform takes advantage of seeding cells in mini-rings of Matrigel® around the rim of 96-well plates. The empty center allows for implementation of liquid handlers and automation, facilitating media exchanges and addition of perturbagens. We kept the empty center feature but altered our geometry to bioprint mini-squares of cells in Matrigel®. By positioning the sides of the square in the imaging path of the HSLCI, we can both sample a larger area as well prevent imaging artifacts due to uneven illumination at edges of the wells. Our bioprinting protocol entails suspending cells in a bioink consisting of a 3:4 ratio of medium and to Matrigel®. This material is then transferred to a print cartridge, incubated at 17° C. for 30 minutes, and bioprinted into each well at a pressure between 12 kPa and 15 kPa.

Using this protocol, standard prints on glass-bottom plates have a thickness of approximately 200 μm. The HSLCI platform uses a wavefront sensing camera and a dynamic focus stabilization system to perform continuous, high-throughput quantitative phase imaging of biological samples, tracking their biomass changes over time. When an object of interest is out of focus, phase information obtained with the interferometric camera cannot be assumed to maintain its direct relationship with the sample's dry biomass.

Thus, by generating thin layers of Matrigel®, we can have a large number of organoids in focus that can be quantitatively assessed at any given time. To generate thinner (˜100 μm) constructs amenable to efficient HSLCI imaging, we increased the hydrophilicity of the glass surface by oxygen plasma treatment. We developed 3D masks composed of BioMed Amber Resin (FormLabs) to selectively functionalize the region of interest. Bioprinting post-plasma treatment generated uniform mini-squares with organoids closely aligned on a single focal plane at (˜70 μm) thickness. We therefore proceeded to verify that thin, printed mini-squares are amenable to massively parallel QPI by HSLCI. Bioprinted organoids can be easily imaged by aligning the legs of the printed mini-square constructs with the HSLCI imaging path.

Lastly, we verified if the printing parameters used altered cell viability by directly comparing MCF-7 cells manually seeded according to cells printed through a 25-gauge needle (260 μm inner diameter) using extrusion pressures ranging from 10 kPa to 25 kPa. We did not observe any reduction in cell viability as measured by ATP release assay. These results are consistent with the existing literature as reductions in cell viability are often associated with higher print pressures (50-300 kPa) which are oftentimes used for extruding more viscous materials. Overall, we have optimized a protocol that generates bioprinted layers suitable for high-throughput HSLCI imaging without impacting cell viability. By having a cell-free well center, the mini-squares retain automation compatibility that is crucial for robust downstream applications.

Bioprinted tumor organoids maintain histological features of manually seeded organoids. Next, we directly compared the histology and immunohistochemical profiles of bioprinted and hand-seeded organoids generated from two breast cancer cell lines, BT-474 and MCF-7, with different molecular features and human epidermal growth factor receptor 2 (HER2) and estrogen receptor (ER) status. We seeded cells as maxi-rings (100,000 cells/ring) to retain sufficient material for downstream characterization. Cells were either manually seeded into 24-well plates or in 8-well plates at an extrusion pressure of 15 kPa. The bioprinted cells and resulting organoid structures are morphologically indistinguishable from the manually seeded counterparts as visible in brightfield images and H&E-stained sections taken 1, 24 and 72 hours after seeding. Both bioprinted and manually seeded samples grew in size over time. Bioprinting did not alter proliferation (Ki-67 staining) or apoptosis (cleaved caspase-3). Hormone receptor status was unaltered as shown by IHC for HER2 and ER. These results are in agreement with literature reports of receptor status for both cell lines. Overall, bioprinting did not influence histologic features.

Bioprinted and manually seeded organoids are molecularly indistinguishable. To further support our findings that the bioprinting protocol we implemented has minimal impacts on the organoids, we also analyzed the transcriptome of manually seeded and bioprinted cells 1 hours, 24 hours, and 72 hours post-seeding. We assessed the distributions of 27,077 RNAs and clustered these into deciles based on their median abundance and found no significant difference in distribution between cell seeding approaches. The overall transcriptomes of manually seeded and bioprinted organoids were extremely well-correlated. Similarly, no individual transcripts differed significantly in RNA abundance in either cell line (0/27,077 genes, q<0.1, Mann-Whitney U-test).

Transcript abundances can be unchanged, but variations in pre-mRNA alternative splicing events can induce functional changes. We found that the density of exon-inclusion and exon-skipping isoforms was unchanged, with no individual fusion isoforms associated with the organoid printing method in either cell line (0/8,561, q<0.1, Mann-Whitney U-test). Similarly, the number of fusion transcripts were not associated with seeding method (p=0.17, Mann-Whitney U-test), although with large numbers of singletons detected in only a small number of samples. Finally, we considered RNA editing; again, we found no significant differences in the number of RNA editing sites between printed and manually developed tumor organoids (p=0.48, Mann-Whitney U-test). These findings demonstrate that our bioprinting protocol does not significantly impact RNA expression, splicing, fusions, or editing sites on short or longer timescales, preserving their molecular profiles while introducing favorable features like reduced thickness suitable for high-throughput imaging using HSLCI.

Trends in mass accumulation of bioprinted organoids can be quantified by HSLCI with single organoid resolution. The full organoid screening pipeline includes cell bioprinting (day 0), organoid establishment (day 0-2), full media replacement (day 3) followed by transfer to the HSLCI incubator. Within 6 hours of media exchange, the plates are continuously imaged for the following 48-72 hours. At the end of the imaging period, we perform an endpoint ATP assay to assess cell viability.

Using HSLCI-based imaging allowed real-time tracking of n=67 MCF-7 organoids in n=8 interpretable replicate wells (8.38/well) and n=101 BT-474 organoids in n=12 interpretable wells (8.42/well) 6 hours after treatment. After 48 hours, the number of tracked organoids increased marginally for both MCF-7 (n=89 organoids; n=8 wells; 11.13/well) and BT-474 organoids (n=106 organoids; n=10 wells; 10.6/well). Over the entire imaging period, n=219 MCF-7 and n=265 BT-474 organoids were tracked for at least six hours. The number of organoids tracked can be improved by using position-specific reference images during pre-processing, analyzing more fields of view within each well, and implementing different image segmentation and tracking algorithms.

Next, we determined the mass of each organoid by converting interferograms to phase shift images using the SID4 software development kit. Organoid mass was calculated by integrating the phase shift over the organoid area and multiplying by the experimentally determined specific refractive increment. At the beginning of the imaging period, the average organoid mass was slightly larger for MCF-7 (2.0±1.2 ng) than BT-474 organoids (1.6±0.5 ng). The difference persisted after 48 hours with MCF-7 organoids averaging 2.5±1.9 ng and BT-474 organoids growing to 2.4±1.0 ng. BT-474 cells grew at a rate of 1.01±3.13% per hour while MCF-7 organoids demonstrated slower average hourly growth rates (0.23±2.92% per hour). The growth rate of the 3D BT-474 organoids is comparable to that observed after 6 hours in 2D culture (approximately 1.3%), while the MCF-7 organoids showed a lower growth rate than previously reported 2D cultures (approximately 1.7%). We also observed a positive association between initial organoid mass and growth rate (95% confidence interval of slope is 0.1040 to 0.2993), but only for MCF-7 organoids.

While the average parameters quantified above offer a population-wide picture of organoid behavior, the power of HSLCI imaging is its ability to quantify intra-sample heterogeneity. We identified several mechanisms by which organoids gained mass over time, including cell growth, cell division, and/or the aggregation of multiple cells or small organoids. We quantified the ratio of organoids that gained, lost, and maintained mass over 12, 24, and 48 hours. In the absence of drug treatment, 1.9% of BT-474 organoids lost more than 10% of their initial mass and 82.1% gained more than 10% of their initial mass over 48 hours. In contrast, only 37.1% of MCF-7 organoids gained mass and 20.2% lost mass. The heterogeneity in the organoid populations also becomes evident over time as 20.4% of MCF-7 organoids gained more than 10% mass within 12 hours. This proportion nearly doubles to 36.7% after 24 hours but remains consistent with a marginal increase to 37.1% after 48 hours. This pattern differs from BT-474 organoids as the population of organoids that gained mass continually increases over 48 hours. The proportion of organoids that gained >10% mass increases from 31.2% after 12 hours, to 61.8% after 24 hours, and 82.1% after 48 hours. Overall, our data confirms that HSLCI can be used to image tumor organoids in 3D, and to quantify both population- and single organoid-level characteristics and heterogeneity.

TABLE 1

| | | | | Organoid Behavior (%) | | |
|---|---|---|---|---|---|---|
| | | Time | | Gained | | Lost |
| | | (h) | n | Mass | Stable | Mass |
| MCF-7 | | | | | | |
| Vehicle | 1% DMSO | 12 | 93 | 20.4 | 74.2 | 5.4 |
| | | 24 | 109 | 36.7 | 51.4 | 11.9 |
| | | 48 | 89 | 37.1 | 42.7 | 20.2 |
| Staurosporine | 0.1 μM | 12 | 34 | 8.8 | 76.5 | 14.7 |
| | | 24 | 43 | 16.3 | 60.5 | 23.3 |
| | | 48 | 34 | 29.4 | 38.2 | 32.4 |
| | 1 μM | 12 | 28 | 7.1 | 82.1 | 10.7 |
| | | 24 | 28 | 3.6 | 71.4 | 25.0 |
| | | 48 | 30 | 0/0 | 73.3 | 26.7 |
| | 10 μM | 12 | 49 | 2.0 | 79.6 | 18.4 |
| | | 24 | 37 | 0.0 | 51.4 | 48.6 |
| | | 48 | 28 | 3.6 | 32.1 | 64.3 |
| Lapatinib | 0.1 μM | 12 | 34 | 17.6 | 73.5 | 8.8 |
| | | 24 | 32 | 28.1 | 56.3 | 15.6 |
| | | 48 | 32 | 46.9 | 34.4 | 18.8 |
| | 1 μM | 12 | 34 | 8.8 | 88.2 | 2.9 |
| | | 24 | 31 | 45.2 | 45.2 | 9.7 |
| | | 48 | 38 | 55.3 | 26.3 | 18.4 |
| | 10 μM | 12 | 44 | 6.8 | 84.1 | 9.1 |
| | | 24 | 46 | 26.1 | 56.5 | 17.4 |
| | | 48 | 37 | 32.4 | 32.4 | 35.1 |
| | 50 μM | 12 | 29 | 6.9 | 58.6 | 34.5 |
| | | 24 | 28 | 14.3 | 39.3 | 46.4 |
| | | 48 | 23 | 4.3 | 43.5 | 52.2 |
| BT-474 | | | | | | |
| Vehicle | 1% DMSO | 12 | 125 | 31.2 | 65.6 | 3.2 |
| | | 24 | 136 | 61.8 | 33.1 | 5.1 |
| | | 48 | 106 | 82.1 | 16.0 | 1.9 |
| Staurosporine | 0.1 μM | 12 | 58 | 27.6 | 70.7 | 1.7 |
| | | 24 | 72 | 45.8 | 50.0 | 4.2 |
| | | 48 | 77 | 70.1 | 23.4 | 6.5 |
| | 1 μM | 12 | 42 | 19.0 | 47.6 | 33.3 |
| | | 24 | 44 | 13.6 | 29.5 | 56.8 |
| | | 48 | 25 | 4.0 | 12.0 | 84.0 |
| | 10 μM | 12 | 42 | 7.1 | 64.3 | 28.6 |
| | | 24 | 46 | 19.6 | 23.9 | 56.5 |
| | | 48 | 27 | 14.8 | 18.5 | 66.7 |
| Lapatinib | 0.1 μM | 12 | 64 | 28.1 | 68.8 | 3.1 |
| | | 24 | 57 | 49.1 | 45.6 | 5.3 |
| | | 48 | 31 | 67.7 | 25.8 | 6.5 |
| | 1 μM | 12 | 54 | 14.8 | 77/8 | 7.4 |
| | | 24 | 59 | 20.3 | 54.2 | 25.4 |
| | | 48 | 40 | 27.5 | 50.0 | 22.5 |
| | 10 μM | 12 | 56 | 19.6 | 73.2 | 7.1 |
| | | 24 | 63 | 9.5 | 60.3 | 30.2 |
| | | 48 | 53 | 15.1 | 47.2 | 37.7 |
| | 50 μM | 12 | 65 | 13.8 | 67.7 | 18.5 |
| | | 24 | 72 | 9.7 | 55.6 | 34.7 |
| | | 48 | 26 | 3.8 | 11.5 | 84.6 |

Time-dependent differences in drug response of organoids can by quantified by HSCLI. We then tested the utility of our platform in detecting drug responses in high-throughput 3D screenings. As proof-of-principle we tested staurosporine, a non-selective protein kinase inhibitor with broad cytotoxicity, and lapatinib, a targeted small molecule tyrosine kinase inhibitor targeting EGFR and HER2. Drugs were tested at concentrations between 0.1 and 50 μM. This range includes and extends beyond the maximum plasma concentration reported for lapatinib (4.2 μM). Representative HSLCI images demonstrate a range of responses to treatment. The average masses at 6 hours post-treatment are not significantly different from the vehicle control. After 48 hours, we observed significant differences in a number of treated samples. Control MCF-7 organoids averaged 2.48±1.89 ng, while those treated with 1 μM and 10 μM staurosporine showed significant reductions in average masses to 1.33±1.08 ng (p=0.0121, Dunnett's multiple comparisons test) and 1.26±0.80 ng (p=0.0086, Dunnett's multiple comparisons test), respectively. BT-474 organoids showed a similar pattern with control organoids averaging 2.36±1.02 ng and staurosporine-treated organoids averaging 0.70±0.26 ng (1 μM, p<0.0001, Dunnett's multiple comparisons test) and 0.83±0.72 ng (10 μM, p<0.0001, Dunnett's multiple comparisons test). The normalized growth curves rapidly show response to treatment with 1 μM staurosporine. After 12 hours, 33.3% of the tracked BT-474 organoids lost mass compared to 3.2% in the control, with a 60% reduction in the number of organoids that increased in size (31.2% vs 19%).

Responses to lapatinib distinctly showed cell type-specific trends. MCF-7 cells exhibited a significant reduction in average mass (from 2.48±1.89 ng to 1.32±1.06 ng, p=0.0285, Dunnett's multiple comparisons test) at 48 h only when treated with 50 μM of lapatinib. Conversely, BT-474 were affected at concentrations as low as 1 μM. Our analysis shows that 4.3% of all MCF-7 organoids continue to grow in the presence of 50 μM of lapatinib and an additional 43.5% maintained their mass after 48 hours of treatment. BT-474 organoids show a dose-dependent response when treated with lapatinib, with concentrations of 0.1, 1, 10 and 50 μM leading to 6.5%, 22.5%, 37.7%, and 84.6% of all BT-474 organoids losing mass (vs 1.9% for controls). The heightened sensitivity of BT-474 cells to lapatinib is expected given the higher expression of HER2 found in these cells.

We consistently observed a fraction of organoids that do not respond to treatment across all conditions. For instance, a fraction of the MCF-7 organoids treated with 50 μM lapatinib on average grew at similar rates as vehicle-treated cells after 36 h of treatment. While overall more sensitive to lapatinib, we could also pinpoint individual BT-474 organoids that did not respond. Our findings are indicative of a resistant population of organoids that can be rapidly identified by HSLCI imaging.

To validate the responses measured by HSLCI, we performed an endpoint ATP-release assay on the same plates used for HSLCI imaging to assess organoid viability after 72 h of treatment. The ATP assay confirmed that both cell lines are highly sensitive to staurosporine treatment with near-zero viability when treated with 1 μM and 10 μM concentrations. Additionally, BT-474 organoids show significant reductions in viability when treated with 0.1 μM lapatinib for 72 hours. Overall, the results of the cell viability assay after 72 hours confirm the trends observed in as little as 12 hours by HSLCI.

TABLE 2

| Vehicle | Staurosporine | | | Lapatinib | | | |
|---|---|---|---|---|---|---|---|
| 1% DMSO | 0.1 μM | 1 μM | 10 μM | 0.1 μM | 1 μM | 10 μM | 50 μM |

Mean mass of MCF-7 organoids

| | | | | | | | | | |
|---|---|---|---|---|---|---|---|---|---|
| 6 | Mean | 2.07 ± 1.24 | 1.71 ± 0.90 | 1.77 ± 1.31 | 1.63 ± 0.82 | 2.00 ± 1.43 | 2.20 ± 1.33 | 2.30 ± 1.48 | 1.71 ± 0.84 |
| hours | mass ± SD | | | | | | | | |
| | p-value | — | 0.143 | 0.328 | 0.050 | 0.823 | 0.657 | 0.416 | 0.274 |
| 48 | Mean | 2.48 ± 1.88 | 1.68 ± 1.03 | 1.33 ± 1.07 | 1.26 ± 0.78 | 2.76 ± 2.35 | 2.71 ± 2.09 | 2.46 ± 1.90 | 1.32 ± 1.03 |
| hours | mass ± SD | | | | | | | | |
| | p-value | — | 0.022 | 0.002 | 0.001 | 0.498 | 0.550 | 0.953 | 0.006 |

Mean mass of BT-474 organoids

| | | | | | | | | | |
|---|---|---|---|---|---|---|---|---|---|
| 6 | Mean | 1.62 ± 0.57 | 1.63 ± 0.85 | 1.38 ± 0.49 | 1.46 ± 0.72 | 1.84 ± 1.21 | 1.58 ± 0.78 | 1.74 ± 0.88 | 1.58 ± 0.70 |
| hours | mass ± SD | | | | | | | | |
| | p-value | — | 0.873 | 0.076 | 0.228 | 0.133 | 0.764 | 0.296 | 0.722 |
| 48 | Mean | 2.36 ± 1.02 | 2.12 ± 1.06 | 0.70 ± 0.26 | 0.83 ± 0.72 | 2.33 ± 1.58 | 1.48 ± 0.66 | 1.42 ± 0.75 | 0.92 ± 0.48 |
| hours | mass ± SD | | | | | | | | |
| | p-value | — | 0.117 | 4.5 * 10⁻¹³ | 2.1 * 10⁻¹¹ | 0.890 | 1.1 * 10⁻⁶ | 1.7 * 10⁻⁸ | 1.2 * 10⁻¹⁰ |

Comparisons of cell viability measures by ATP detection assay

| | | | | | | | | | |
|---|---|---|---|---|---|---|---|---|---|
| 6 | Mean | 1.00 ± 0.21 | 0.50 ± 0.08 | 0.17 ± 0.04 | 0.13 ± 0.03 | 1.00 ± 0.03 | 1.01 ± 0.04 | 0.66 ± 0.07 | 0.14 ± 0.03 |
| hours | mass ± SD | | | | | | | | |
| | p-value | — | 1.1 * 10⁻⁵ | 5.6 * 10⁻⁹ | 2.2 * 10⁻⁹ | 0.972 | 0.926 | 0.023 | 2.0 * 10⁻⁵ |
| 48 | Mean | 1.00 ± 0.20 | 0.58 ± 0.14 | 0.02 ± 0.01 | 0.01 ± 0.00 | 0.70 ± 0.18 | 0.43 ± 0.09 | 0.32 ± 0.04 | 0.09 ± 0.07 |
| hours | mass ± SD | | | | | | | | |
| | p-value | — | 2.8 * 10⁻⁴ | 1.2 * 10⁻⁹ | 1.0 * 10⁻⁹ | 0.009 | 1.3 * 10⁻⁵ | 1.1 * 10⁻⁶ | 3.4 * 10⁻⁸ |

TABLE 3
Organoid viability analysis by endpoint ATP assay

| | | | MCF-7 | | BT-474 | |
|---|---|---|---|---|---|---|
| | | | Viability ± SD | p-value | Viability ± SD | p-value |
| Vehicle | 1% DMSO | | 1.00 ± 0.21 | | 1.00 ± 0.20 | |
| Staurosporine | 0.1 | μM | 0.50 ± 0.08 | <0.0001 | 0.58 ± 0.14 | 0.0002 |
| | 1 | μM | 0.17 ± 0.04 | <0.0001 | 0.02 ± 0.01 | <0.0001 |
| | 10 | μM | 0.13 ± 0.03 | <0.0001 | 0.01 ± 0.00 | <0.0001 |
| Lapatinib | 0.1 | μM | 1.00 ± 0.03 | 0.9452 | 0.70 ± 0.18 | 0.0113 |
| | 1 | μM | 1.01 ± 0.04 | 0.8598 | 0.43 ± 0.09 | <0.0001 |
| | 10 | μM | 0.66 ± 0.07 | 0.0018 | 0.32 ± 0.04 | <0.0001 |
| | 50 | μM | 0.14 ± 0.03 | <0.0001 | 0.09 ± 0.07 | <0.0001 |

Chemotherapies, targeted therapies or biologics can be screened using the platform. Drugs such but not limited to as staurosporine, docetaxel, gemcitabine, temozolomide, larotrectinib, ibrutinib, lapatinib, vandetanib, pazopanib, bortezomib, panobinostat, linsitinib, capivastertib, sunitinib, everolimus, lenvatinib, regorafenib, carfilzomib, imatinib, crizotinib, pembrolizumab, nivolumab, ipilimumab, trastuzumab, and cabozantinib may be screened. Additionally, combinatorial therapies such as but not limited to gemcetabine and docetaxel, carfilzomib and panabinostat, bortezomib and panobinostat, sorafenib and everolimus, and cabozantinib and everolimus may be evaluated.

The above screening platform can be expanded to include an immune system component to evaluate intrinsic immune reactivity and screen immunotherapy agents, either alone or in combination with targeted agents or chemotherapeutics. This platform can be used to integrate the organoid screening platform with assay cells obtained from the same patient. In any embodiment, the assay cells may be immune cells. In one embodiment, the immune cells co-cultured with the tumor organoids can be tumor infiltrating lymphocytes (TILs) that are isolated from the tumor from the patient. In another embodiment, the immune cells can be sorted circulating immune cells isolated from the patient. Co-cultures can be established by adding the isolated immune cells in solution or by embedding them in tissue-like gel matrices extruded by a bioprinter.

In one embodiment, the population of assay cells comprise two or more cell types, in separate locations or combined together. In one embodiment, the population of assay cells is provided in the tissue culture well. In one embodiment, the population of assay cells comprise two or more cell types. Any combination or location or any one or more assay cell types is embraced herein.

In some embodiments, all features, uses, methods, arrangements, locations, and other descriptions provided herein wherein the tumor organoids are provided in the shaped organoid extrudate on the well bottom, and the assay cells provided in the well, are interchangeable. In some embodiments, the motility and/or viability and/or function of immune cells provided in the shaped organoid extrudate are imaged or otherwise characterized to assess characteristics as described herein.

In one embodiment, immune cell functions of the co-cultured immune cells such as cytokine production or immune cell growth can be determined by techniques generally known in the art. In another embodiment, cell number or cell growth of CD4 and/or CD8 T cells, as well as that of the organoid, can be determined using microscopy and/or metabolic readouts. In another embodiment, cellular viability can be determined using microscopy and/or metabolic readouts.

The method described above can be used to screen antitumor and/or immunoregulatory compounds to determine their ability to enhance T-cell anti-tumor effects. In one embodiment, the platform described herein can be used to perform IO combinatorial screenings with small molecules that are either targeted agents or chemotherapy regimens. In one embodiment, the platform and system described herein can be used to perform rapid, highly personalized screenings to identify efficacious IO regimens. Cancer-immune cells co-culture growth conditions can be determined by methods and techniques generally known in the art. Tumor organoids can be obtained from a large number of different tumor types, e.g., sarcoma or lung tumors can be routinely obtained through generally available sources and techniques.

In one embodiment, the platform described above can be used to screen immune checkpoint inhibitor therapies, such as nivolumab, pembrolizumab, cemiplimab, atezolizumab, avelumab, dervalumab, or ipilimumab. In another embodiment, the platform can be used to screen cell therapies such as CAR-T cell therapies or NK cell therapies. Combinations of multiple types of therapies can also be investigated, for example nivolumab and rapamycin.

1. A method for identifying therapeutic agents or a combination thereof for treating a tumor in a patient, comprising the steps of
i) obtaining a sample of tumor cells in single cell suspension or as aggregates or clusters from the tumor of the patient;
ii) extruding a collection of said tumor cells into tissue culture wells, such that the tumor cells form one or more shaped, three-dimensional organoid extrudates comprising said tumor cells;
iii) co-culturing said shaped organoid extrudate with a population of assay cells in said tissue culture wells in the presence of therapeutic agents or combination thereof, wherein reduced tumor cell functions or increased assay cell functions in the presence of said therapeutic agents or combination thereof identifies the therapeutic agents or combination thereof for treating the tumor in the patient.

2. A method for bioprinting biological samples by extruding a collection of cells in a hydrogel into tissue culture wells, such that the cells form one or more geometrically-shaped, three-dimensional extrudates.

3. The method of claim 1, wherein said tumor cell functions comprise tumor cell growth, tumor cell viability or tumor cell mobility.

4-6. (canceled)

7. The method of claim 1, wherein the assay cells are immune cells.

8. The method of claim 6, wherein said immune cell functions comprise cytokine production or immune cell growth.

9. The method of claim 1, wherein the assay cells are liver cells.

10. (canceled)

11. The method of claim 1, wherein said shaped organoid extrudates are deposited as rings around a perimeter at a bottom of said tissue culture wells.

12. The method of claim 2, wherein said shaped cell extrudates are deposited as rings around a perimeter at a bottom of said tissue culture wells.

13-16. (canceled)

17. The method of claim 1, wherein said shaped organoid extrudates comprise a hydrogel.

18. (canceled)

19. The method of claim 1, wherein the extruding of the tumor cells is performed manually or by automated bioprinting.

20-35. (canceled)

36. The method of claim 1, wherein said assay cells are obtained from said patient.

37. The method of claim 1, wherein said assay cells are circulating immune cells or tumor infiltrating immune cells.

38-39. (canceled)

40. The method of claim 1, wherein said assay cells comprise a hydrogel.

41. (canceled)

42. The method of claim 1, wherein said therapeutic agents comprise chemotherapeutic agents or immunotherapeutic agents.

43. A method for treating a patient having a tumor, comprising the steps of:
i) carrying out the steps of claim 1; and
ii) treating the patient with the therapeutic agents or combination thereof.

44-56. (canceled)

57. An automated method for bioprinting shaped organoid extrudates, comprising the steps of
i) obtaining a tissue sample from a subject and preparing a single cell suspension, cell aggregates or cell clusters of said tissue sample;
ii) mixing said single cell suspension, aggregates or clusters with a gel precursor solution, thereby forming a cell complex comprising cells and a matrix;
iii) dispensing said cell complex into one or more tissue culture wells by one or more dispensers of an automatic device, said dispensers have been calibrated so as to deposit said cell complex as a polygon or ring on a base of said tissue culture wells.

58. The method of claim 57, wherein the dispensing of the cell complex is performed at a temperature of between about 0° C. and about 37° C.

59. (canceled)

60. The method of claim 57, wherein the dispensing of the cell complex is performed through an orifice of about 0.1 to about 1 mm in diameter.

61-65. (canceled)

66. The method of claim 57, wherein the dispensing of the cell complex is performed at an extrusion pressure of between about 1 and about 150 kPa.

67-68. (canceled)

69. The method of claim 57, wherein the dispensing of the cell complex is performed at a printhead speed of from about 1 to about 200 mm/second.

70-76. (canceled)

77. The method of claim 57, wherein said matrix of said cell complex is a hydrogel.

78-79. (canceled)

80. The method of claim 57, wherein said tissue sample is a tumor sample.

81-90. (canceled)

91. The method of 57, wherein the gel precursor solution comprises a basement membrane extract, the dispensing is at a temperature between about 2 and about 37° C., the print surface temperature is between about 4 and about 37° C., the extrusion pressure is between about 1 and about 25 kPa, and the printhead speed is between about 5 and about 40 mm/s.

92. (canceled)

93. The method of any one of claim 1, 2 or 57, wherein the gel precursor solution comprises CELLINK LAMININK, the dispensing is at a temperature between about 15 and about 25° C., the print surface temperature is between about 20 and about 25° C., the extrusion pressure is between about 35 and about 55 kPa, and the printhead speed is between about 10 and about 20 mm/s.

94. The method of claim 57, wherein the gel precursor solution comprises PPO, the dispensing is at a temperature between about 20 and about 25° C., the print surface temperature is between about 20 and about 25° C., the extrusion pressure is between about 35 and about 150 kPa, and the printhead speed is between about 1 and about 80 mm/s.
